(19) United States
(12) Patent Application Publication
(10) Pub. No.: US 2024/0343868 A1
(43) Pub. Date: Oct. 17, 2024

(54) METHODS OF CROSSLINKING POLYMERS AND HYDROGEL MICROPARTICLES AND OF ENCAPSULATING BIOLOGICALLY ACTIVE COMPOUNDS, COMPOSITIONS MADE THEREFROM, AND DEVICES

(71) Applicant: President and Fellows of Harvard College, Cambridge, MA (US)

(72) Inventors: Daniele FORESTI, Somerville, MA (US); Nils PASQUERO, Cambridge, MA (US); Jennifer A. LEWIS, Cambridge, MA (US)

(73) Assignee: President and Fellows of Harvard College, Cambridge, MA (US)

(21) Appl. No.: 18/580,416

(22) PCT Filed: Jul. 21, 2022
(86) PCT No.: PCT/US22/37837

§ 371 Date: Jan. 18, 2024

Related U.S. Application Data
(60) Provisional application No. 63/224,126, filed on Jul. 21, 2021.

(51) Int. Cl. C08J 3/24; A61K 9/16; B01J 13/14; B33Y 30/00; B33Y 70/00; C08B 37/00

(52) U.S. Cl. CPC C08J 3/24; A61K 9/1652; B01J 13/14; B33Y 70/00; C08B 37/0084; B33Y 30/00; C08J 2305/04

(57) ABSTRACT

A composition including a particle including a crosslinked matrix, prepared from a liquid droplet including a formulation including a polymer, the gelled particle having an average diameter of less than 250 pm, is provided herein. A method of printing the composition including releasing the liquid droplet into a fluid including an aerosol including a crosslinking agent and contacting the liquid droplet with the fluid is further provided. A device configured to introduce an aerosol including a crosslinking agent to a droplet of a formulation including a polymer as the droplet passes through the aerosol including the crosslinking agent is further provided.

### RELATED APPLICATIONS

This application claims the benefit of priority of U.S. Patent Application No. 63/224,126, filed on Jul. 21, 2021, which is incorporated by reference herein in its entirety.

### TECHNICAL FIELD

The present disclosure is related generally to microparticle production and more specifically to crosslinked microparticle compositions.

### BACKGROUND

Hydrogels have become essential tools in tissue engineering, regenerative medicine, and drug delivery, owing to their high water content and biocompatibility. Hydrogel microparticles in particular are seeing increased interest as delivery vehicles of drugs and cells, and as building blocks of macroscale granular structures. Their multiscale properties from the nanoscale (mesh size, electrostatic interactions, to the microscale (particle size and mechanical properties), and the macroscale (interparticle interactions) provide unprecedented freedom in the design of biomaterial-based approaches for biomedical applications. In addition, hydrogel microparticles can be easily injected through needles and catheters due to their micron size, making them highly suited to in vivo administration. The hydrogel microparticles can be loaded with a variety of fragile biologics, such as therapeutic proteins, for local delivery. The modularity and potential of hydrogel microparticle-based systems reside in the ability to tune their properties at the micron scale, i.e., at the microparticle scale. The modulation of these properties may require changing the material composition and concentration or varying the microparticle production parameters.

Microparticles produced conventionally can be produced only from a limited range of materials, and tend to have high polydispersities and non-uniform shapes. Conventional production technologies may rely on high shear stresses, which can be detrimental to hydrogel microparticles with fragile and expensive cargo. Moreover, emulsion-based approaches, including droplet-based microfluidics, may expose the cargos to hydrophobic carrier fluids that can damage the molecules. Photoinitiators used to cure hydrogels may generate radicals upon ultraviolet exposure that are usually toxic. Existing limitations make current technologies ill-suited for the generation and preparation of hydrogel-antibody droplets that may be crosslinked into uniform microparticles, underscoring the need for new manufacturing technologies.

### SUMMARY

In an example, the present disclosure provides a method of preparing a particle. The method includes releasing a liquid droplet from a droplet generator into a first fluid including an aerosol including a crosslinking agent, the droplet including a formulation including a polymer; contacting the liquid droplet with the crosslinking agent in the first fluid; crosslinking the liquid droplet to form the particle including a crosslinked matrix; and collecting the particle.

In another example, the present disclosure provides a composition. The composition includes a particle formed from a liquid droplet including a formulation including a polymer, the particle including a crosslinked matrix. The particle includes a particle surface including a plurality of surface pores, the plurality of surface pores having an average valley depth and/or an average diameter of from about 0.1 μm to about 20 μm.

In yet another example, the present disclosure provides an in-flight crosslinking device. The in-flight crosslinking device includes an aerosol generator configured to introduce into the device a fluid including an aerosol including a crosslinking agent. The in-flight crosslinking device further includes a droplet generator configured to release into the fluid a liquid droplet including a polymer, the liquid droplet contacting the crosslinking agent. The in-flight crosslinking device further includes a target configured to receive an at least partially crosslinked particle formed from the liquid droplet.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

In order that the present disclosure may be well understood, there will now be described various forms thereof, given by way of example, reference being made to the accompanying drawings. The components in the figures are not necessarily to scale.

FIG. 1 illustrates a side view of the flight path of droplets of an example of a formulation through a fluid including an example of a crosslinking agent to a collection bath below, prepared according to the principles of the present disclosure;

FIG. 1A illustrates an exploded view of the indicated portion of FIG. 1;

FIG. 1B illustrates an exploded view of the indicated portion of FIG. 1A;

FIG. 2 illustrates a side view of an example of a MIST In-flight Crosslinking (MISTIC) device during preparation of an example of crosslinked hydrogel microparticles according to the principles of the present disclosure;

FIG. 3 illustrates a perspective view of another example of a MISTIC device;

FIG. 4 illustrates a side view of another example of a MISTIC device;

FIG. 5 illustrates examples of impact behaviors of hydrogel microparticles of different Weber numbers in collection baths, the microparticles prepared according to the principles of the present disclosure;

FIGS. 6A, 6B, 6C, 6D, and 6E illustrate bright-field microscopic image views of examples of microparticles prepared with and without using MISTIC with various polymer concentrations, surface tensions, and biological cargo concentrations, at either 0.1% w/v calcium chloride crosslinking agent concentration (top), or 1.0% w/v calcium chloride crosslinking agent concentration (bottom), with exploded view insets, the microparticles prepared according to the principles of the present disclosure;

FIG. 7 illustrates a bright-field microscopic image view of an example of spherical alginate microparticles with antibody cargo, prepared according to the principles of the present disclosure;

FIG. 8 illustrates a microparticle diameter distribution plot including a gaussian distribution overlay, the microparticles prepared according to the principles of the present disclosure;

FIGS. 9A, 9B, and 9C illustrate bright-field microscopic image views of examples of microparticles prepared without using MISTIC, using oxidized and non-oxidized alginate and calcium chloride crosslinking agent at 1% and 10% w/v, prepared according to the principles of the present disclosure;

FIG. 10 illustrates an example of a mixing nozzle used in the preparation of hydrogel microparticles prepared according to the principles of the present disclosure;

FIGS. 11A, 11B, 11C, and 11D illustrate bright-field microscopic image views of an example of microparticles including click functionalized alginate polymers at various times over the course of five hours, according to the principles of the present disclosure;

FIG. 12 illustrates bright-field microscopic image views of examples of microparticles at three different concentrations of calcium chloride crosslinking agent in the collection bath (10.0% w/v, top; 1.0% w/v, middle; 0.1% w/v, bottom) taken at 0 minutes (left), 3 hours (middle), and 3 weeks (right) in the collection bath;

FIG. 13 illustrates a side view of yet another example of a MISTIC device including an example of a droplet generated outside of an example of a fluid including an aerosol including a crosslinking agent and an example of a crosslinked particle collected outside the example of the fluid, according to the principles of the present disclosure;

FIG. 13A illustrates a side view of yet another example of a MISTIC device including an example of a droplet generator spaced apart from an example of a fluid and an example of a collection target spaced apart from the example of the fluid, according to the principles of the present disclosure;

FIG. 13B illustrates a side view of yet another example of a MISTIC device including an example of a droplet generator in an example of a fluid;

FIG. 13C illustrates a side view of yet another example of a MISTIC device including an example of a droplet generator and a target in an example of a fluid;

FIG. 13D illustrates a side view of yet another example of a MISTIC device including an example of a target in an example of a fluid;

FIG. 13E illustrates a comparison of examples of MISTIC devices including a static aerosol and dynamic aerosols;

FIG. 13F illustrates examples of MISTIC devices including different directions of droplet release into aerosols and different directions of droplet trajectory through the aerosols;

FIG. 14 illustrates the effect of a change in width in an example of a chamber of an example of a MISTIC device;

FIG. 15A illustrates a perspective view of an example of a MISTIC device including a plurality of droplets released simultaneously from a droplet generator; and

FIG. 15B illustrates a perspective view of an example of a MISTIC device including a plurality of droplets released simultaneously from a plurality of droplet generators.

The drawings described herein are for illustration purposes only and are not intended to limit the scope of the present disclosure in any way.

### DETAILED DESCRIPTION

The following description is merely exemplary in nature and is not intended to limit the present disclosure, application, or uses.

In the present disclosure, a method of preparing a particle of uniform size and shape, with tunable surface roughness and microparticle shrinkage characteristics, is provided. The method is characterized by contacting a liquid droplet with a first fluid comprising an aerosol comprising a crosslinking agent. The liquid droplet includes a formulation including a polymer. As the liquid droplet passes through the first fluid, the liquid droplet forms the particle comprising a crosslinked matrix, and the particle is received by a target. The method enables preparation of generally monodisperse particles of average diameters of less than 250 μm, and preferably less than 100 μm, with a coefficient of variation of less than about 4.0%. Further, the particles prepared by the methods described herein are generally spherical, without the tail defects observed in the absence of the “in-flight” crosslinking.

In certain examples, the liquid droplet is released from a droplet generator, which may include a nozzle. In other examples, the polymer may be a hydrogel precursor. In still other examples, the particle may be a gelled particle including a crosslinked hydrogel matrix. In still other examples, the formulation may further include a cargo, and the crosslinked matrix may include the cargo dispersed in the crosslinked matrix. In still other examples, the cargo may be a biologic. In still other examples, the target is a collection bath, which may include a solution of the crosslinking agent.

Further, the methods disclosed herein enable the generation of microparticles composed of high concentrations of polymers and biological cargos (biologics). The microparticle manufacturing technology disclosed herein is characterized by the absence of both high shear forces and hydrophobic carrier fluids, which is believed to be essential for encapsulating high viscosity formulations of active proteins and other biological cargos.

In the present disclosure, a device is provided to contact a falling droplet with an aerosol including a crosslinking agent prior to the droplet landing in a collection bath. The in-flight crosslinking device ensures the preparation of gelled particles of uniform spherical shape and size of less than about 250 μm average diameter.

### Methods of Preparation of Droplets Including Formulations

Methods for preparing compositions suitable for subcutaneous or intravenous delivery of a therapeutic agent are described herein. Examples of compositions suitable for subcutaneous or intravenous delivery may include a liquid droplet prior to crosslinking and a gelled particle after crosslinking, where the liquid droplet includes a formulation including a polymer, and the gelled particle includes a crosslinked matrix. Notably, in certain examples the formulation may have a relatively high viscosity in a range from about mPa·s to about 500,000 mPa·s. In other examples, the viscosity may be at least about 100 MPa·s, at least about 200 mPa·s, at least about 500 mPa·s, or at least about 1000 mPa·s, and is typically about 400,000 mPa·s, or about 200,000 mPa·s or less.

Without limitation, in examples, droplets including a formulation including the hydrogel precursor and the biological cargo may be prepared by a variety of methods. Examples of methods of preparing formulations may include: spraying, acoustophoretic printing, ink jet printing, solenoidal valve printing, co-flow printing, electrohydrodynamic printing, dynamic printing, and transfer printing.

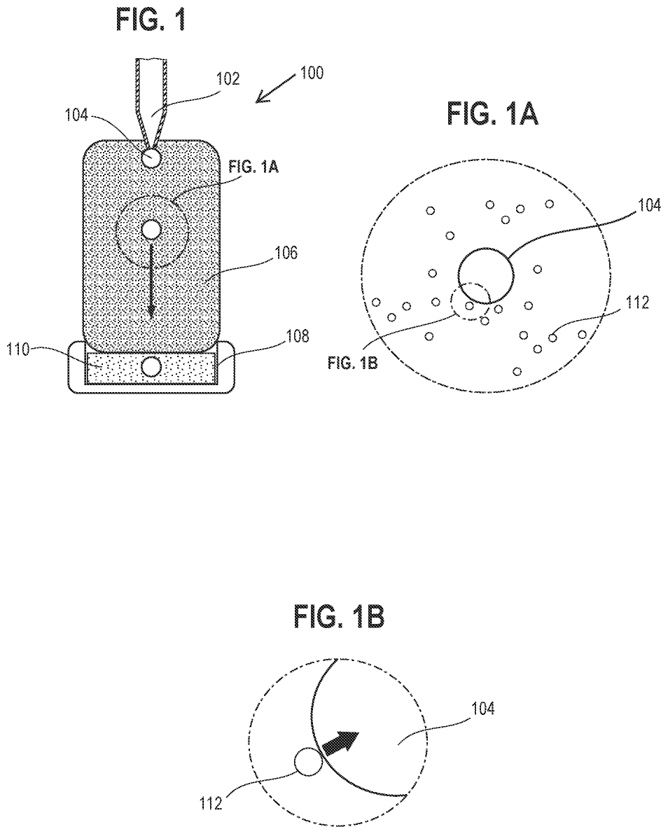

Referring to FIG. 1, in an example, using an apparatus 100, the preparation of a crosslinked hydrogel matrix may include the preparation of a droplet 104 including a formulation, droplet 104 released in a first fluid 106 as a liquid droplet from a droplet generator 102, and droplet 104 undergoing crosslinking to form a gelled particle including a crosslinked hydrogel matrix with the biological cargo dispersed therein. More specifically, the hydrogel precursor in the droplet 104 undergoes crosslinking to form the crosslinked hydrogel matrix. The crosslinking may be initiated by a crosslinking reagent. The crosslinking may take place before or after the liquid droplet 104 is deposited on a substrate or enters a liquid bath 108, which may include a crosslinking solution 110. Referring now to FIG. 1A, in certain examples, the crosslinking may take place in the first fluid, which may include the crosslinking agent 112 in an aerosol, prior to or after reaching the substrate or liquid bath 108. In other examples, the crosslinking may occur in a liquid bath. As illustrated in FIG. 1B, the crosslinking agent 112 collides with the droplet 104.

In another example, a method may include: releasing a liquid droplet from a droplet generator into a first fluid including an aerosol including a crosslinking agent, the droplet including a formulation including a polymer; contacting the liquid droplet with the crosslinking agent in the first fluid; crosslinking the liquid droplet to form a particle including a crosslinked matrix; and collecting the particle.

In certain examples, the method may further include: generating an acoustic field in the first fluid with an oscillating emitter; and detaching the droplet from the droplet generator by acoustic forces from the acoustic field.

In certain examples, the particle may be a gelled particle including a crosslinked hydrogel matrix, and the polymer may be a hydrogel precursor.

In certain examples, the formulation of the liquid droplet may include a cargo, and the cargo may be dispersed in the crosslinked matrix. In other examples, the cargo is a biologic. In still other examples, the cargo may be homogeneously dispersed in the crosslinked matrix.

In certain examples, the method may be performed continuously, such as by releasing a continuous stream of droplets from the nozzle opening over a predetermined period of time.

In other examples, the method may further include flowing air with the liquid droplet during the releasing in order to increase the frequency of droplet release. In further examples, so as to optimize the contacting of the liquid droplet with the crosslinking agent, the fluid may flow in a direction and speed similar to the direction and speed of the liquid droplet, with at most a small relative velocity between the fluid and the liquid droplet.

In certain examples, the first fluid may be air. In other examples, the aerosol may include an aqueous solution of the crosslinking agent.

In certain examples, the crosslinking may include forming a crosslinked shell prior to the collecting.

In certain examples, the contacting, crosslinking, and/or collecting may be performed in the absence of ultraviolet radiation.

In certain examples, the collecting of the particles may be in a collection bath, which may include a solution of the crosslinking agent.

In an example, the formulation may include the polymer at a concentration of at least about 10 mg/mL, at least about 20 mg/mL, at least about 50 mg/mL, at least about 100 mg/mL, or at least about 200 mg/mL, and/or as high as about 1000 mg/mL, as high as about 800 mg/mL, as high as about 600 mg/mL, or as high as about 500 mg/mL. In certain examples, the formulation may also or alternatively include the cargo at a concentration of at least about 10 mg/mL, at least about 20 mg/mL, at least about 50 mg/mL, at least about 100 mg/mL, or at least about 200 mg/mL, and/or as high as about 1000 mg/mL, as high as about 800 mg/mL, as high as about 600 mg/mL, or as high as about 500 mg/mL. Preferably, in certain examples, the formulation may have a pH below an isoelectric point of the cargo, although in some examples the formulation may have a pH above the isoelectric point.

In an example, an excipient may be included in the formulation. In certain examples, the excipient may stabilize the cargo (for example, protein). Examples of excipients may include one or more of the following: a buffering agent, such as citrate, phosphate, acetate, and/or histidine buffer; an amino acid, such as L-arginine hydrochloride and/or L-glutamic acid; an antioxidant, such as ascorbic acid, methionine, and/or ethylenediaminetetraacetic acid (EDTA); a surfactant, such as Polysorbate 80, Polysorbate 20, Brij 30, Brij 35, and/or Pluronic F127; a preservative, such as benzyl alcohol, cresol, phenol, and/or chlorobutanol.

In an example, an adjuvant, which may trigger an immune reaction, may be included in the formulation. An adjuvant may be beneficial for vaccine delivery. Examples of adjuvants may include one or more of the following: an aluminum salt, such as amorphous aluminum hydroxyphosphate sulfate (AAHS), aluminum hydroxide, aluminum phosphate, potassium aluminum sulfate; and/or cytosine phosphoguanine (CpG).

Examples of crosslinking agents 112 may include calcium chloride (for example, 0.1 wt. %) adjusted to a suitable pH, for example, with sodium hydroxide or with a chitosan (for example, 0.25 wt. %) and acetic acid mixture. The suitable pH of the crosslinking agent may be below an isoelectric point of the biological cargo. As described above, the formulation including the polymer and the cargo may also have a pH below or above the isoelectric point of the cargo. Further, the formulation may include the hydrogel precursor at a concentration of at least about 20 mg/mL and/or as high as about 100 mg/mL. Also, or alternatively, the formulation may include the cargo at a concentration of at least about 20 mg/mL, at least about 50 mg/mL, at least about 100 mg/mL, at least about 150 mg/mL, at least about 200 mg/mL, at least about 250 mg/mL, or at least about 300 mg/mL and/or as high as about 700 mg/mL, as high as about 750 mg/mL, as high as about 800 mg/mL, as high as about 850 mg/mL, as high as about 900 mg/mL, as high as about 950 mg/mL, or as high as about 1000 mg/mL. The particles may remain in the liquid bath for a time duration of from about 30 minutes to about 90 minutes.

In certain examples, the crosslinking agent may be present in the first fluid and/or in the collection bath in a concentration of from about 0.1% w/v, or from about 0.2% w/v, or from about 0.3% w/v, or from about 0.4% w/v, or from about 0.5% w/v, or from about 0.6% w/v, or from about 0.7% w/v, or from about 0.8% w/v, or from about 0.9% w/v, or from about 1.0% w/v, or from about 1.5% w/v, or from about 2.0% w/v, or from about 2.5% w/v, or from about 3.0% w/v, or from about 3.5% w/v, or from about 4.0% w/v, or from about 4.5% w/v, or from about 5.0% w/v, or from about 5.5% w/v, or from about 6.0% w/v, or from about 6.5% w/v, or from about 7.0% w/v, or from about 7.5% w/v, or from about 8.0% w/v, or from about 8.5% w/v, or from about 9.0% w/v, or from about 9.5% w/v to about 10.0% w/v; or from about 0.1% w/v to about 0.2% w/v, or to about 0.3% w/v, or to about 0.4% w/v, or to about 0.5% w/v, or to about 0.6% w/v, or to about 0.7% w/v, or to about 0.8% w/v, or to about 0.9% w/v, or to about 1.0% w/v, or to about 1.5% w/v, or to about 2.0% w/v, or to about 2.5% w/v, or to about 3.0% w/v, or to about 3.5% w/v, or to about 4.0% w/v, or to about 4.5% w/v, or to about 5.0% w/v, or to about 5.5% w/v, or to about 6.0% w/v, or to about 6.5% w/v, or to about 7.0% w/v, or to about 7.5% w/v, or to about 8.0% w/v, or to about 8.5% w/v, or to about 9.0% w/v, or to about 9.5% w/v; or from any one of the above minima to any one of the above maxima.

By the methods described herein, examples of monodisperse microparticles of an unprecedented range of concentrations (for example, 2.5-10% w/w) and viscosities (above 200-15,000 cP) may be produced on demand with potentially zero waste and independently of flow rate.

### MISTIC Device

In an example, an in-flight crosslinking device may include an aerosol generator configured to introduce into the device a fluid including an aerosol including a crosslinking agent; a droplet generator configured to release into the fluid a liquid droplet including a polymer, the liquid droplet contacting the crosslinking agent; and a target configured to receive an at least partially crosslinked particle formed from the liquid droplet.

In certain examples, an in-flight crosslinking device may include a chamber configured to confine the fluid, the chamber including an opening through which the liquid droplet is released into the fluid and a second opening through which the target receives the at least partially crosslinked particle. In other examples, the aerosol generator may be configured to flow the fluid into the chamber. In still other examples, the aerosol generator may be configured to flow the fluid in a direction parallel to a trajectory of the liquid droplet. In still other examples, the aerosol generator may be configured to flow the fluid in a direction at an angle to a trajectory of the liquid droplet.

In certain examples, the chamber may be generally in the shape of a cone. In other examples, the chamber may be generally cylindrical. In still other examples, the chamber increases in width from the opening to the second opening.

In certain examples, the target may include a collector. In other examples, the target may include a collection bath.

In certain examples, the target may be spaced apart from the fluid. In other examples, the droplet generator may be spaced apart from the fluid.

In certain examples, the chamber may be configured to block ultraviolet radiation.

In certain examples, the droplet generator may be configured to release a plurality of droplets into the fluid simultaneously. In other examples, the plurality of droplets may be released simultaneously from a plurality of droplet generators. In still other examples, the droplet generator includes a mixing nozzle. In still other examples, the droplet generator may be an acoustophoretic printer.

In certain examples, the aerosol generator may be configured to introduce the fluid at a volumetric flow rate such that the fluid flows through the device at a speed identical to which the liquid droplet passes through the device.

In an example, a device includes a chamber including an inlet configured to introduce into the chamber a fluid comprising an aerosol including a crosslinking agent; an outlet configured to evacuate the fluid from the chamber; a top opening through which a droplet generator projects into the chamber; and a bottom opening configured to seal around a collection container open to the chamber. The collection container may be configured to contain a solution including the crosslinking agent. The chamber is configured to contact a droplet of a formulation including a polymer released from the droplet generator with the aerosol as the droplet passes through the chamber and before the droplet lands in the solution. In certain examples, the device may include a source of forced gas and be configured to flow the gas in a direction parallel to the direction of the release of the droplet from the droplet generator and with the droplet as the droplet passes through the chamber.

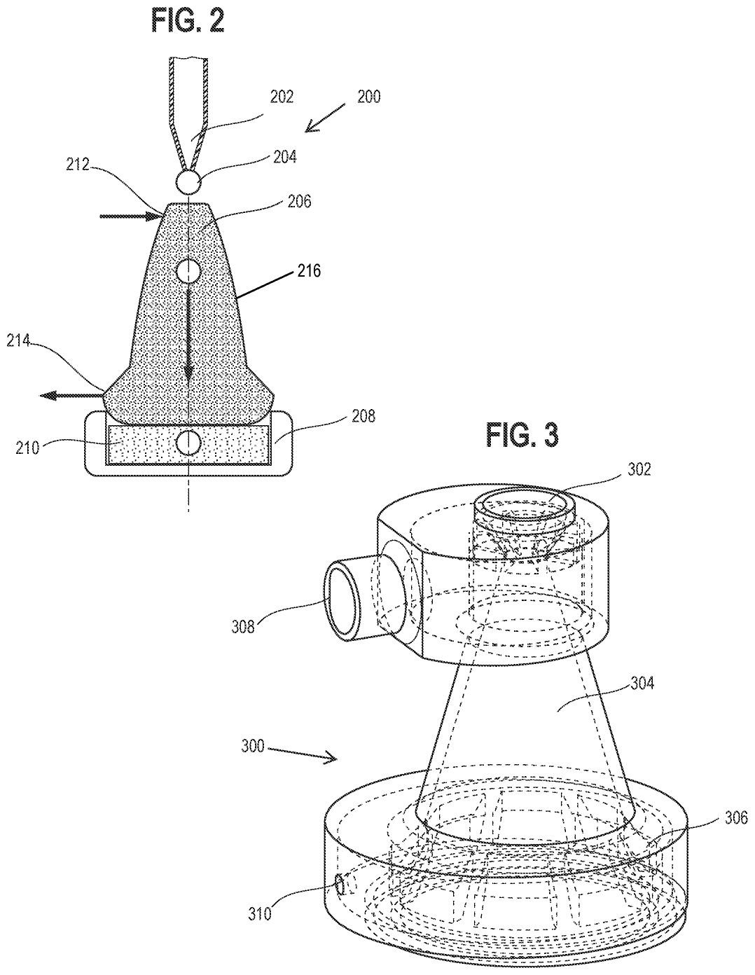

Referring now to FIG. 2, an example of a MISTIC device includes a chamber 216 generally in the shape of a cone. A sidewall of chamber 216 includes an inlet 212 at the top of the chamber 216 configured to introduce a first fluid including a crosslinking aerosol 206, and an outlet 214 at the bottom of the chamber 216 configured to evacuate the crosslinking aerosol 206. The chamber 216 includes an opening through which a droplet 204 may be ejected from a droplet generator, and pass through the crosslinking aerosol 206 and out through a hole at the bottom of chamber 216 into a collection bath 208 including a second fluid 210. The chamber 216 may be fabricated from a material that blocks ultraviolet radiation. Chamber 216 may be an output of a three-dimensional printer.

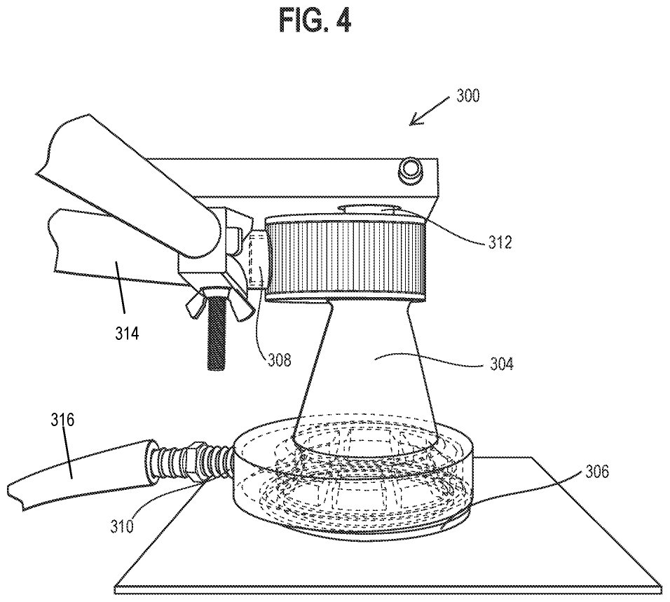

In another example, as illustrated in FIG. 3, top opening 302 of MISTIC device 300 may be advantageously conically shaped, and the crosslinking aerosol may be introduced through inlet 308 into a space around the top opening that is fluidly connected to chamber 304. The chamber 304 may be further fluidly connected to a ring-shaped base that covers collection bath 306, the base including outlet 310 that is configured to evacuate the crosslinking aerosol. As illustrated in FIG. 4, MISTIC device 300 is connected to droplet generator 312, inlet hose 314 that may be fluidly connected to a moisturizer (not shown) configured to generate the crosslinking aerosol, and outlet hose 316 that may be fluidly connected to a vacuum (not shown) configured to evacuate the chamber.

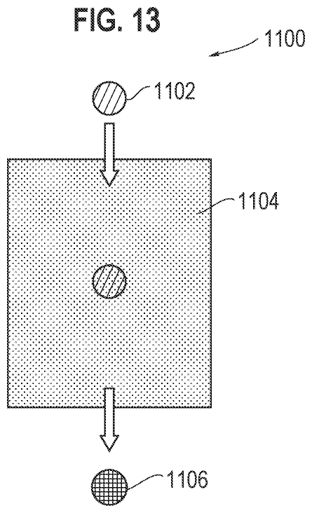

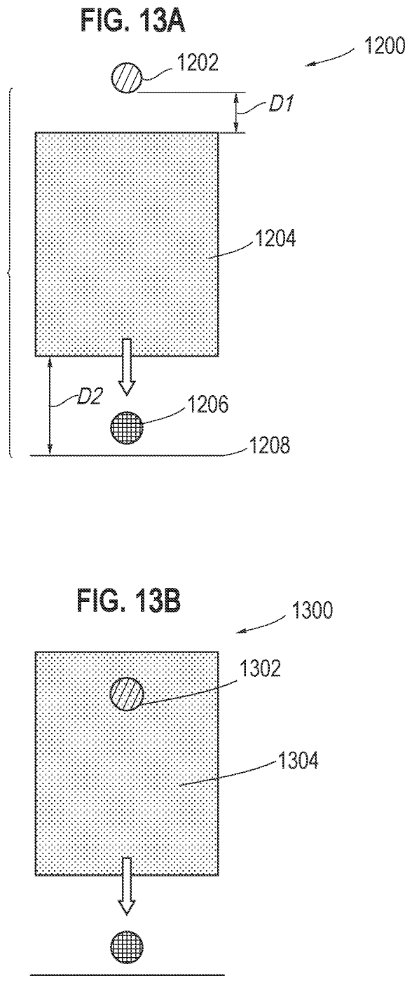

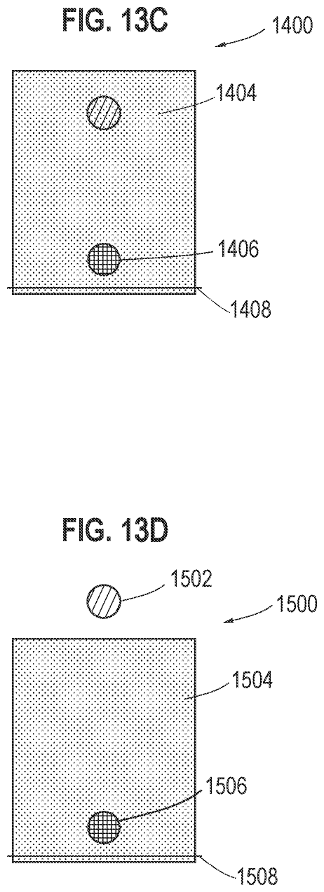

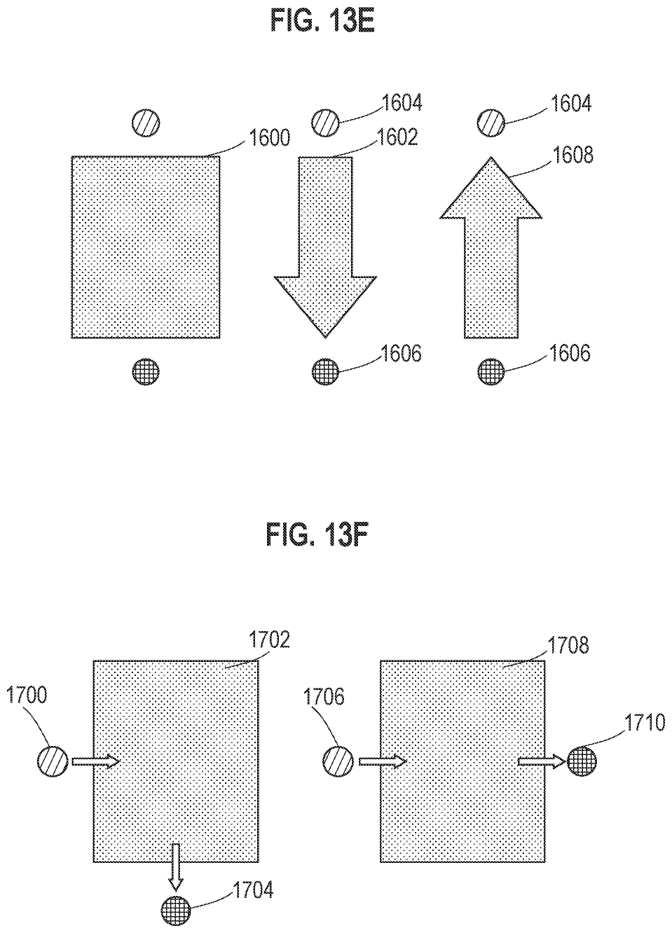

Referring to FIG. 13, a side view of another example of an in-flight crosslinking device 1100 is illustrated. In in-flight crosslinking device 1100, liquid droplet 1102 is generated outside of fluid 1104. Fluid 1104 includes an aerosol including a crosslinking agent. Fluid 1104 may be in a chamber configured to confine fluid 1104. Liquid droplet 1102 contacts the aerosol in fluid 1104 and exits fluid 1104 as an at least partially crosslinked particle 1106 to be received by a target.

Referring to FIG. 13A, a side view of another example of in-flight crosslinking device 1200 is illustrated. In in-flight crosslinking device 1200, liquid droplet 1202 is generated outside of fluid 1204 by a droplet generator (not shown) that is spaced apart from fluid 1204 by distance D1. Distance D1 may be any distance, without limitation. Fluid 1204 includes an aerosol including a crosslinking agent. Fluid 1204 may be in a chamber configured to confine fluid 1204. Liquid droplet 1202 contacts the aerosol in fluid 1204 and exits fluid 1204 as an at least partially crosslinked particle 1206 to be received by target 1208. Target 1208 may be spaced apart from fluid 1204 by distance D2. Distance D2 may be any distance, without limitation.

In an example, distance D1 may be in a range of from about 1 μm to about 1 m. In certain examples, distance D1 may be in a range of from about 10 μm, or from about 20 μm, or from about 30 μm, or from about 40 μm, or from about 50 μm, or from about 60 μm, or from about 70 μm, or from about 80 μm, or from about 90 μm, or from about 100 μm, or from about 200 μm, or from about 300 μm, or from about 400 μm, or from about 500 μm, or from about 600 μm, or from about 700 μm, or from about 800 μm, or from about 900 μm, or from about 1 mm, or from about 5 mm, or from about 1 cm, or from about 2 cm, or from about 3 cm, or from about 4 cm, or from about 5 cm, or from about 6 cm, or from about 7 cm, or from about 8 cm, or from about 9 cm, or from about 10 cm, or from about 20 cm, or from about 30 cm, or from about 40 cm, or from about 50 cm, or from about 60 cm, or from about 70 cm, or from about 80 cm, or from about 90 cm, to about 1 m; or from about 1 μm to about 10 μm, or to about 20 μm, or to about 30 μm, or to about 40 μm, or to about 50 μm, or to about 60 μm, or to about 70 μm, or to about 80 μm, or to about 90 μm, or to about 100 μm, or to about 200 μm, or to about 300 μm, or to about 400 μm, or to about 500 μm, or to about 600 μm, or to about 700 μm, or to about 800 μm, or to about 900 μm, or to about 1 mm, or to about 5 mm, or to about 1 cm, or to about 2 cm, or to about 3 cm, or to about 4 cm, or to about 5 cm, or to about 6 cm, or to about 7 cm, or to about 8 cm, or to about 9 cm, or to about 10 cm, or to about 20 cm, or to about 30 cm, or to about 40 cm, or to about 50 cm, or to about 60 cm, or to about 70 cm, or to about 80 cm, or to about 90 cm; or from any one of the above minima to any one of the above maxima. In certain examples, a particularly preferred range for distance D1 is from about 1 mm to about 10 mm.

In an example, distance D2 may be in a range of from about 1 μm to about 1 m. In certain examples, distance D2 may be in a range of from about 10 μm, or from about 20 μm, or from about 30 μm, or from about 40 μm, or from about 50 μm, or from about 60 μm, or from about 70 μm, or from about 80 μm, or from about 90 μm, or from about 100 μm, or from about 200 μm, or from about 300 μm, or from about 400 μm, or from about 500 μm, or from about 600 μm, or from about 700 μm, or from about 800 μm, or from about 900 μm, or from about 1 mm, or from about 5 mm, or from about 1 cm, or from about 2 cm, or from about 3 cm, or from about 4 cm, or from about 5 cm, or from about 6 cm, or from about 7 cm, or from about 8 cm, or from about 9 cm, or from about 10 cm, or from about 20 cm, or from about 30 cm, or from about 40 cm, or from about 50 cm, or from about 60 cm, or from about 70 cm, or from about 80 cm, or from about 90 cm, to about 1 m; or from about 1 μm to about 10 μm, or to about 20 μm, or to about 30 μm, or to about 40 μm, or to about 50 μm, or to about 60 μm, or to about 70 μm, or to about 80 μm, or to about 90 μm, or to about 100 μm, or to about 200 μm, or to about 300 μm, or to about 400 μm, or to about 500 μm, or to about 600 μm, or to about 700 μm, or to about 800 μm, or to about 900 μm, or to about 1 mm, or to about 5 mm, or to about 1 cm, or to about 2 cm, or to about 3 cm, or to about 4 cm, or to about 5 cm, or to about 6 cm, or to about 7 cm, or to about 8 cm, or to about 9 cm, or to about 10 cm, or to about 20 cm, or to about 30 cm, or to about 40 cm, or to about 50 cm, or to about 60 cm, or to about 70 cm, or to about 80 cm, or to about 90 cm; or from any one of the above minima to any one of the above maxima. In certain examples, a particularly preferred range for distance D2 is from about 1 mm to about 10 cm.

Referring to FIG. 13B, a side view of another example of in-flight crosslinking device 1300 is illustrated. In in-flight crosslinking device 1300, liquid droplet 1302 is generated inside fluid 1304.

Referring to FIG. 13C, a side view of another example of in-flight crosslinking device 1400 is illustrated. At least partially crosslinked particle 1406 is received by target 1408 inside fluid 1404 including an aerosol including a crosslinking agent.

Referring to FIG. 13D, a side view of another example of in-flight crosslinking device 1500 is illustrated. In in-flight crosslinking device 1500, liquid droplet 1502 is generated outside of fluid 1504. At least partially crosslinked particle 1506 is received by target 1508 inside fluid 1504.

Referring to FIG. 13E, a comparison between in-flight crosslinking devices including a static fluid 1600 and dynamic fluids 1602, 1608 is illustrated. The static fluid 1600 and dynamic fluids 1602, 1608 each include an aerosol including a crosslinking agent. In the example shown in FIG. 13E, dynamic fluid 1602 may flow in the same direction as liquid droplet 1604, which passes through dynamic fluid 1602 and exits as at least partially crosslinked particle 1606. Alternatively, in another example, dynamic fluid 1608 may flow in a direction opposite that of liquid droplet 1604.

Referring to FIG. 13F, a comparison between in-flight crosslinking devices including different directions of liquid droplets 1700, 1706 being released into fluids 1702, 1708 respectively, is illustrated. In an example, liquid droplet 1700 is released at a substantially horizontal angle into fluid 1702, and at least partially crosslinked particle 1704 exits from fluid 1702 at an angle substantially perpendicular to the angle at which liquid droplet 1700 is released into fluid 1702. By contrast, in another example, liquid droplet 1706 is released at a substantially horizontal angle into fluid 1708, and at least partially crosslinked particle 1710 exits from fluid 1708 at the same substantially horizontal angle.

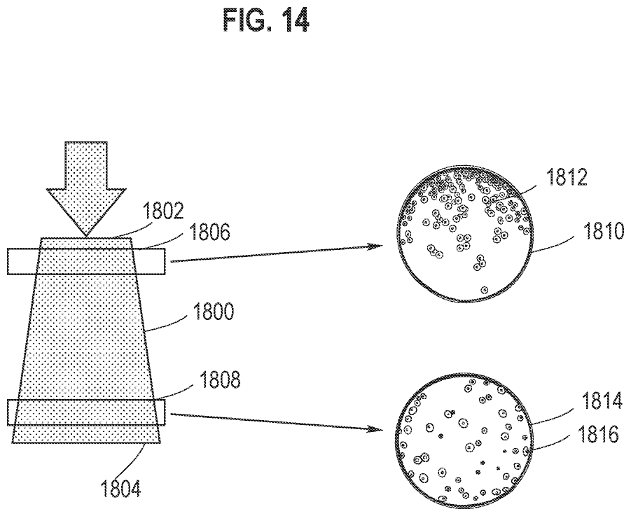

Referring to FIG. 14, chamber 1800 of an example of an in-flight crosslinking device includes a top opening 1802 and a bottom opening 1804, with the width of chamber 1800 increasing from top opening 1802 to bottom opening 1804. Consequently, a width 1806 of chamber 1800 that is closer to top opening 1802 is smaller than a second width 1808 of chamber 1800 that is closer to bottom opening 1804. Due to the smaller width 1806, the speed of flow of the fluid in chamber 1800 is higher, and the crosslinking agent within the aerosol within the fluid collides with liquid droplet 1810 at a higher frequency at the top of liquid droplet 1810, resulting in more impacts 1812 towards the top of liquid droplet 1810, and an unevenly distributed surface roughness. By contrast, the larger width 1808 results in a lower speed of flow of the fluid in chamber 1800, and the crosslinking agent within the aerosol within the fluid collides with liquid droplet 1814 at a higher frequency at the bottom of liquid droplet 1814, resulting in more impacts 1816 toward the bottom of liquid droplet 1814.

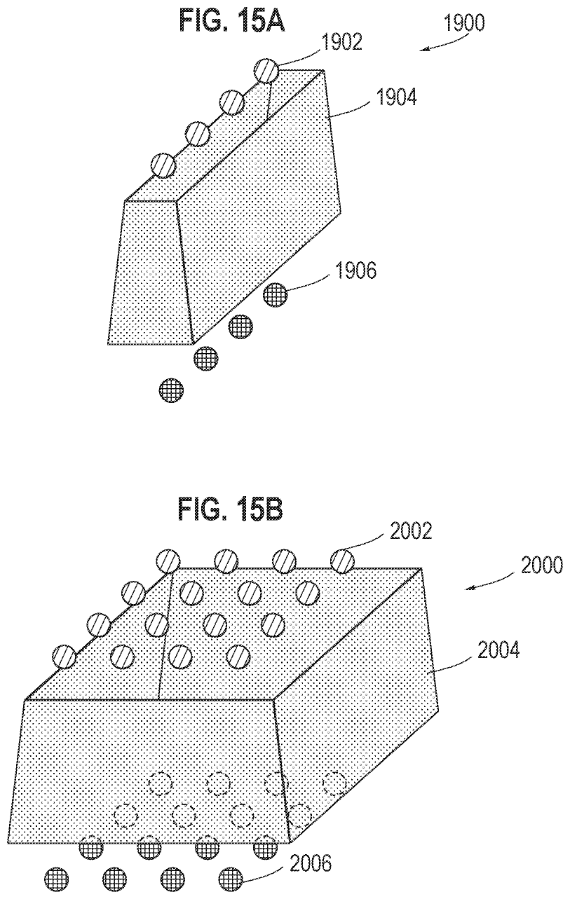

Referring to FIG. 15A, an example of an in-flight crosslinking device 1900 is illustrated. In-flight crosslinking device 1900 includes chamber 1904 sized to contact a plurality of droplets 1902 with an aerosol including a crosslinking agent, the aerosol within a fluid confined by chamber 1904. The plurality of droplets 1902 are released simultaneously from a droplet generator and exit chamber 1904 as a plurality of at least partially crosslinked particles 1906.

Referring to FIG. 15B, an example of an in-flight crosslinking device 2000 is illustrated. In-flight crosslinking device 2000 includes chamber 2004 sized to contact a plurality of droplets 2002 with an aerosol including a crosslinking agent, the aerosol within a fluid confined by chamber 2004. The plurality of droplets 2002 are released simultaneously from a plurality of droplet generators and exit chamber 2004 as a plurality of at least partially crosslinked particles 2006.

### Definitions

The following definitions are included to provide a clear and consistent understanding of the specification and claims. As used herein, the recited terms have the following meanings. All other terms and phrases used in this specification have their ordinary meanings as one of skill in the art would understand.

References in the specification such as “one example” or “an example” indicate that the example described may include a particular aspect, feature, or characteristic, but not every example necessarily includes that aspect, feature, or characteristic. Moreover, such phrases may, but do not necessarily, refer to the same example referred to in other portions of the specification. Further, when a particular aspect, feature, or characteristic is described in connection with an example, it is within the knowledge of one skilled in the art to affect or connect such aspect, feature, or characteristic with other examples, whether or not explicitly described.

The singular forms “a,” “an,” and “the” include plural referents unless the context clearly dictates otherwise. Thus, for example, a reference to “a microparticle” includes a plurality of such microparticles. It is further noted that the claims may be drafted to exclude any optional element. As such this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The terms “one or more” and “at least one” are readily understood by one of skill in the art, particularly when read in context of its usage. For example, the phrase may mean one, two, three, four, five, six, ten, 100, or any upper limit approximately 10, 100, or 1000 times higher than a recited lower limit.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably unless indicated otherwise herein. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than or less than a recited integer at each end of the range. Unless indicated otherwise herein, the terms “about and “approximately” are intended to include, for example, weight percentages, proximate to the recited range, that are equivalent in terms of the functionality of the individual ingredient, composition, or example. The terms “about” and “approximately” can also modify the end-points of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if “10 to 15” is disclosed, then 11, 12, 13, and 14 are also disclosed, individually, and as part of a range. A recited range (for example, weight percentages) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third, and upper third. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within broader ratio. Accordingly, specific values recited for ranges are for illustration only; they do not exclude other defined values or other values within defined ranges. It will be further understood that the endpoints of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members in a recited group. Accordingly, provisos may apply to any of the disclosed categories or examples whereby any one or more of the recited elements, species, or examples, may be excluded from such categories or examples, for instance, for use in an explicit negative limitation.

The term “coefficient of variation” (CV) refers to the ratio of the standard deviation to the mean and shows the extent of variability in a set of values in relation to the mean. The lower the CV, the more monodisperse the set of values. The greater the CV, the more polydisperse the set of values.

The term “sphericity” refers to the ratio of the surface area of a perfect sphere to the surface area of a spherically shaped object, such as a microparticle.

The term “aspect ratio,” with respect to a spherically shaped object, such as a microparticle, refers to the ratio of the radius of the object from the center point along the x- or y-axis relative to the radius of the object from the center point along the z-axis.

The term “dimensional Weber particle number” (We) refers to the ratio between the particle inertia and the surface tension, as detailed in formula (3) below:

$$We = ( Inertial forces ) / ( Surface tension ) = [ ρ \cdot {( {u}_{d} )}^{2} \cdot D ] / σ \qquad ( 3 )$$

where ρ is the solution density, u_{d} is the droplet velocity, D is the droplet diameter, and a is the surface tension of the bath solution.

The term “surface roughness” refers to the presence of a plurality of surface pores in a surface of a particle.

The term “click chemistry” refers to a class of small molecule reactions in which substrates are joined quickly and irreversibly in one pot in high chemical yield with high reaction specificity. The reactions are typically insensitive toward oxygen and water and have a large thermodynamic driving force (such as greater than 20 kcal/mol) favoring a reaction with a single reaction product. Examples of click chemical reactions may include copper(I)-catalyzed azide-alkyne cycloaddition, strain-promoted azide-alkyne cycloaddition, strain-promoted alkyne-nitrone cycloaddition, alkene and azide [3+2] cycloaddition, alkene and tetrazine inverse-demand Diels-Alder, and alkene and tetrazole photoclick reaction.

### Acoustophoretic Printing

In acoustophoretic printing, acoustic waves are exploited to generate a net force on a pendant drop. In particular, the nonlinear effect of the acoustic field—namely radiation pressure—is able to exert a surface force surface Fₐ at the droplet interface (typically in addition to the gravity force F_{g}) so as to overcome the capillary force Fc. The equation for the nonlinear effect can be written as formula (1) below:

$${F}_{c} = π σ d = {F}_{g} + {F}_{a} = V ρ ( g + {g}_{a} ) \qquad ( 1 )$$

where F_{c}=rad is the capillary force for a given liquid with surface tension σ, which opposes both the gravity force F_{g}=(⅙)πD³ μg=Vμg, where D is the drop diameter, V is the drop volume, ρ is the fluid density, and g is gravitational acceleration. The acoustophoretic acceleration gₐ embeds all the nonlinear effects of the acoustic field and its modeling into a single parameter, and I is measured in units of g=9.81 m·s⁻². The parameter gₐ scales with the square of the acoustic pressure P, and may be represented by gₐ∝P². P may be controlled by controlling the voltage of the sound source. By increasing gₐ, the droplet volume at detachment V may be linearly decreased, as illustrated in formula (2) below:

$$V = π d σ / ρ ( g + {g}_{a} ) \qquad ( 2 )$$

In an example of the formulation including the hydrogel precursor and the biological cargo prepared by acoustophoretic printing, the formulation including the hydrogel precursor and the biological cargo may be flowed through a nozzle, the nozzle typically having an outer diameter d of from about 50 to 100 μm. The formulation is ejected from the nozzle into a fluid and/or a bath that may contain a crosslinking fluid. The formulation flow rate may be constant or variable, and may be in a range of from greater than 0 to 150 microliters per minute. The airborne nature of acoustophoretic printing provides the ability to vary independently different parameters to ensure the production of unique alginate microparticles.

The acoustophoretic force may allow for control over the size of a microparticle. Acoustophoretic printing may produce monodisperse microparticles of different diameters (D=465±13 μm, 407±4 μm, 336±3 μm, 379±7 μm, 215±7 μm, and 176±4 μm) by only controlling the parameter gₐ (14.4 g, 21.4 g, 38.4 g, 67.2 g, 145.4 g, 264.6 g, respectively). Precise control over microparticle size distribution is beneficial for drug delivery kinetics and for good manufacturing practice requirements. Values of gₐ may exceed 250 g due to improved acoustic field resonance. Despite the high viscosity of the 5 wt. % alginate solution (apparent viscosity μ₀=800 mPa·s), the monodispersity is conserved independently of the gₐ applied, with coefficient of variation (CV) below 4%.

Acoustophoretic printing may produce hydrogel microparticles at very high concentrations of alginate (10 wt. %) with viscosity μ₀=15,000 mPa·s and maintain a high level of monodispersity (CV=1.4%).

A key aspect of acoustophoretic printing is the decoupling between flow rate and droplet detachment. Microparticle size distribution may be illustrated to be unaffected by variation in flow rate (CV=3.1%, gₐ=245 g). This quasi-“drop-on-demand” approach may be extremely convenient in microparticle production, making acoustophoretic printing a very robust process for microparticle production. Additionally, acoustophoretic printing eliminates the need for long ramping up time, reaching of equilibrium, or droplet formation.

### Crosslinked Particles

In certain examples, the particles formed according to the present disclosure may have an average diameter of less than 250 μm, or less than 240 μm, or less than 230 μm, or less than 220 μm, or less than 210 μm, or less than 200 μm, or less than 190 μm, or less than 180 μm, or less than 170 μm, or less than 160 μm, or less than 150 μm, or less than 140 μm, or less than 130 μm, or less than 120 μm, or less than 110 μm, or less than 100 μm, or less than 90 μm, or less than 80 μm, or less than 70 μm, or less than 60 μm, or less than 50 μm, or less than 40 μm, or less than 30 μm, or less than 20 μm, or less than 10 μm.

In certain examples, the coefficient of variation (CV) of the average diameter of the crosslinked particles formed according to the present disclosure may be less than 5.0%, or less than 4.9%, or less than 4.8%, or less than 4.7%, or less than 4.6%, or less than 4.5%, or less than 4.4%, or less than 4.3%, or less than 4.2%, or less than 4.1%, or less than 4.0%, or less than 3.9%, or less than 3.8%, or less than 3.7%, or less than 3.6%, or less than 3.6%, or less than 3.5%, or less than 3.4%, or less than 3.3%, or less than 3.2%, or less than 3.1%, or less than 3.0%, or less than 2.9%, or less than 2.8%, or less than 2.7%, or less than 2.6%, or less than 2.5%, or less than 2.4%, or less than 2.3%, or less than 2.2%, or less than 2.1%, or less than 2.0%, or less than 1.9%, or less than 1.8%, or less than 1.7%, or less than 1.6%, or less than 1.5%, or less than 1.4%, or less than 1.3%, or less than 1.2%, or less than 1.1%, or less than 1.0%, or less than 0.9%, or less than 0.8%, or less than 0.7%, or less than 0.6%, or less than 0.5%, or less than 0.4%, or less than 0.3%, or less than 0.2%, or less than 0.1%.

In certain examples, a solution in a collection bath may have a surface tension of about 40 mN/m. In other examples, a solution in a collection bath may have a surface tension of about 10 mN/m, or about 15 mN/m, or about 20 mN/m, or about 25 mN/m, or about 30 mN/m, or about 35 mN/m, or about 40 mN/m, or about 45 mN/m, or about 50 mN/m, or about 55 mN/m, or about 60 mN/m, or about 65 mN/m, or about 70 mN/m, or about 75 mN/m, or about 80 mN/m, or about 85 mN/m.

Examples of the crosslinked particles may be delivered subcutaneously or intravenously into a human body. Also or alternatively, the delivery or administration of examples of the crosslinked particles may include by one or more of the following: uricular, buccal, conjunctival, cutaneous, dental, electro-osmotical, endocervical, endosinusial, endotracheal, enteral, epidural, extra amniotical, extracorporeal, infiltration, interstitial, intra-abdominal, intra-amniotical, intra-arterial, intra-articular, intrabiliary, intrabronchial, intrabursal, intracardial, intracartilaginous, intracaudal, intracavernous, intracavitary, intracerebral, intracisternal, intracorneal, intracoronal, intracoronary, intracorporus cavernosum, intradermal, intradiscal, intraductal, intraduodenal, intradural, intraepidermal, intraesophageal, intragastrical, intragingival, intraileal, intralesional, intraluminal, intralymphatical, intramedullar, intrameningeal, intramuscular, intraocular, intraovarian, intrapericardial, intraperitoneal, intrapleural, intraprostatical, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastrical, occlusive dressing technique, ophthalmical, oral, oropharyngeal, parenteral, percutaneous, periarticular, peridural, perineural, periodontal, rectal, inhalation, retrobulbar, soft tissue, subarachnoidial, subconjunctival, subcutaneous, sublingual, submucosal, topical, transdermal, transmucosal, transplacental, transtracheal, transtympanic, ureteral, urethral, and/or vaginal administration. The crosslinked particles may have an encapsulation efficiency of at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%.

Examples of the crosslinked particles may include a particle surface including a plurality of surface pores, consequently characterizing the particle surface as having “surface roughness.” In certain examples, the plurality of surface pores may have an average valley depth of from about 0.1 μm, or from about 0.2 μm, or from about 0.3 μm, or from about 0.4 μm, or from about 0.5 μm, or from about 0.6 μm, or from about 0.7 μm, or from about 0.8 μm, or from about 0.9 μm, or from about 1.0 μm, or from about 1.5 μm, or from about 2.0 μm, or from about 2.5 μm, or from about 3.0 μm, or from about 3.5 μm, or from about 4.0 μm, or from about 4.5 μm, or from about 5.0 μm, or from about 5.5 μm, or from about 6.0 μm, or from about 6.5 μm, or from about 7.0 μm, or from about 7.5 μm, or from about 8.0 μm, or from about 8.5 μm, or from about 9.0 μm, or from about 9.5 μm, or from about 10.0 μm, or from about 10.5 μm, or from about 11.0 μm, or from about 11.5 μm, or from about 12.0 μm, or from about 12.5 μm, or from about 13.0 μm, or from about 13.5 μm, or from about 14.0 μm, or from about 14.5 μm, or from about 15.0 μm, or from about 15.5 μm, or from about 16.0 μm, or from about 16.5 μm, or from about 17.0 μm, or from about 17.5 μm, or from about 18.0 μm, or from about 18.5 μm, or from about 19.0 μm, or from about 19.5 μm to about 20.0 μm; or from about 0.1 μm to about 0.2 μm, or to about 0.3 μm, or to about 0.4 μm, or to about 0.5 μm, or to about 0.6 μm, or to about 0.7 μm, or to about 0.8 μm, or to about 0.9 μm, or to about 1.0 μm, or to about 1.5 μm, or to about 2.0 μm, or to about 2.5 μm, or to about 3.0 μm, or to about 3.5 μm, or to about 4.0 μm, or to about 4.5 μm, or to about 5.0 μm, or to about 5.5 μm, or to about 6.0 pam, or to about 6.5 μm, or to about 7.0 μm, or to about 7.5 μm, or to about 8.0 μm, or to about 8.5 μm, or to about 9.0 μm, or to about 9.5 μm, or to about 10.0 μm, or to about 10.5 μm, or to about 11.0 μm, or to about 11.5 μm, or to about 12.0 μm, or to about 12.5 μm, or to about 13.0 μm, or to about 13.5 μm, or to about 14.0 μm, or to about 14.5 μm, or to about 15.0 μm, or to about 15.5 μm, or to about 16.0 μm, or to about 16.5 μm, or to about 17.0 μm, or to about 17.5 μm, or to about 18.0 μm, or to about 18.5 μm, or to about 19.0 μm, or to about 19.5 μm, or to about 20.0 μm; or from any one of the above minima to any one of the above maxima.

In certain examples, the plurality of surface pores may have an average diameter of from about 0.1 μm, or from about 0.2 μm, or from about 0.3 μm, or from about 0.4 μm, or from about 0.5 μm, or from about 0.6 μm, or from about 0.7 μm, or from about 0.8 μm, or from about 0.9 μm, or from about 1.0 μm, or from about 1.5 μm, or from about 2.0 μm, or from about 2.5 μm, or from about 3.0 μm, or from about 3.5 μm, or from about 4.0 μm, or from about 4.5 μm, or from about 5.0 μm, or from about 5.5 μm, or from about 6.0 μm, or from about 6.5 μm, or from about 7.0 μm, or from about 7.5 μm, or from about 8.0 μm, or from about 8.5 μm, or from about 9.0 μm, or from about 9.5 μm, or from about 10.0 μm, or from about 10.5 μm, or from about 11.0 μm, or from about 11.5 μm, or from about 12.0 μm, or from about 12.5 μm, or from about 13.0 μm, or from about 13.5 μm, or from about 14.0 μm, or from about 14.5 μm, or from about 15.0 μm, or from about 15.5 μm, or from about 16.0 μm, or from about 16.5 μm, or from about 17.0 μm, or from about 17.5 μm, or from about 18.0 μm, or from about 18.5 μm, or from about 19.0 μm, or from about 19.5 μm to about 20.0 μm; or from about 0.1 μm to about 0.2 μm, or to about 0.3 μm, or to about 0.4 μm, or to about 0.5 μm, or to about 0.6 μm, or to about 0.7 μm, or to about 0.8 μm, or to about 0.9 μm, or to about 1.0 μm, or to about 1.5 μm, or to about 2.0 μm, or to about 2.5 μm, or to about 3.0 μm, or to about 3.5 μm, or to about 4.0 μm, or to about 4.5 μm, or to about 5.0 μm, or to about 5.5 μm, or to about 6.0 pam, or to about 6.5 μm, or to about 7.0 μm, or to about 7.5 μm, or to about 8.0 μm, or to about 8.5 μm, or to about 9.0 μm, or to about 9.5 μm, or to about 10.0 μm, or to about 10.5 μm, or to about 11.0 μm, or to about 11.5 μm, or to about 12.0 μm, or to about 12.5 μm, or to about 13.0 μm, or to about 13.5 μm, or to about 14.0 μm, or to about 14.5 μm, or to about 15.0 μm, or to about 15.5 μm, or to about 16.0 μm, or to about 16.5 μm, or to about 17.0 μm, or to about 17.5 μm, or to about 18.0 μm, or to about 18.5 μm, or to about 19.0 μm, or to about 19.5 μm, or to about 20.0 μm; or from any one of the above minima to any one of the above maxima.

Examples of the crosslinked matrix of the crosslinked particles may include acrylate, acrylonitrile, alginate, agar, agarose, carboxymethylcellulose, carrageenan, chitosan, chondroitin sulfate, collagen, dextran, fibrin, gelatin, hyaluronate, hydroxyethylcellulose, xanthan, polylysine, poly(acrylic) acid, poly(ethylene glycol) and its derivatives, cellulose and its derivatives, poly(propylene glycol) and its derivatives, polylactide and its derivatives, poly(glycolic acid) and its derivatives, poly(propylene fumarate) and its derivatives, polycaprolactone and its derivatives, polyhydroxybutyrate and its derivatives, polyacrylates and derivatives, poly(vinylpyrrolidone) and its derivatives, and/or poly(ethylenimine) and its derivatives. Consistent with this, examples of the polymer employed for the formulation may include an acrylate precursor, an acrylonitrile precursor, alginate precursor, an agar precursor, an agarose precursor, a carboxymethylcellulose precursor, a carrageenan precursor, a chitosan precursor, a chondroitin sulfate precursor, a collagen precursor, a dextran precursor, a fibrin precursor, a gelatin precursor, a hydroxyethylcellulose precursor, a hyaluronate precursor, a xanthan precursor, a polylysine precursor, a poly(acrylic) acid precursor, a precursor for poly(ethylene glycol) and its derivatives, a precursor for cellulose and its derivatives, a precursor for poly(propylene glycol) and its derivatives, a precursor for polylactide and its derivatives, a precursor for poly(glycolic acid) and its derivatives, a precursor for poly(propylene fumarate) and its derivatives, a precursor for polycaprolactone and its derivatives, a precursor for polyhydroxybutyrate and its derivatives, a precursor for polyacrylates and derivatives, a precursor for poly(vinylpyrrolidone) and its derivatives, and/or a precursor for poly(ethylenimine) and its derivatives.

Examples of the biological cargo in the crosslinked particles may include a protein, a checkpoint inhibitor, a hormone, a peptide, a nucleic acid, a mammalian cell, a micro-organism, a small molecule, a bacterium, a drug (for example, an antibody-based drug, such as monoclonal antibodies, antibody-drug conjugates, bispecific antibodies), a cytokine (for example, interleukin, interferon, tumor necrosis factor, chemokine, transforming growth factor beta, growth factor), insulin, Botulinum toxin type A, Botulinum toxin type B, bovine serum albumin (BSA), human immunoglobulin G (IgG), Fc fusion protein, an anticoagulant, a blood factor, a bone morphogenetic protein, an engineered protein scaffold, an enzyme, a thrombolytic, and/or another biological substance.

Advantageously, in certain examples, the cargo may be homogeneously dispersed in the crosslinked matrix. Examples of polymer-to-cargo mass:mass ratios may include a range of from about 10:1 to about 1:1000. In certain examples, the ratio may be at least about 15:1, at least about 10:1, at least about 5:1, at least about 4:1, at least about 3:1, at least about 2:1, at least about 1:1, at least about 1:2, at least about 1:3, at least about 1:4, at least about 1:5, at least about 1:10, at least about 1:20, at least about 1:50, or at least about 1:100, and/or the ratio may be no greater than about 1:1000, no greater than about 1:800, or no greater than about 1:500.

In some examples, a shell may encapsulate the crosslinked particle. Typically, the shell includes a biocompatible polymer. Core-shell structures are a class of particles that are composed of two or more different material layers. One layer forms the inner core and the other layers make the outer layers or the shell. This type of design provides the opportunity to tune the composite material that exhibits characteristics and properties not achievable by the individual materials of the core and the shell. For any type of applications, a well-controlled synthesis of the core-shell microparticles is imperative as the synthesis will directly affect parameters such as size or morphology, and indirectly, such as encapsulation efficiency.

Exemplary biocompatible polymers useful for core-shell structures include chitosan and its derivatives and/or cationic dextran and its derivatives, cationic cellulose and its derivatives, cationic gelatin and its derivatives, Poly(2-N,N-dimethylaminoethylmethacrylate) and its derivatives, poly-L-lysine and its derivatives, polyethyleneimine and its derivatives, poly(amidoamine)s and its derivatives. Typically, the gelled particles may have an average diameter in a range of from about 10 microns to about 2 millimeters, and the gelled particles may be monodisperse, as described below.

The compositions and processes described above may be better understood in connection with the following Examples. In addition, the following non-limiting examples are an illustration. The illustrated methods are applicable to other examples of crosslinking of formulations of hydrogel precursors and biological cargos of the present disclosure. The procedures described as general methods describe what is believed will be typically effective to crosslink formulations of hydrogel precursors and biological cargos indicated. However, the person skilled in the art will appreciate that it may be necessary to vary the procedures for any given example of the present disclosure, for example, vary the order or steps and/or the chemical reagents used.

### Examples

### A. Materials and Methods

### 1. Hydrogel Precursor Solutions.

Alginate was purchased from Sigma-Aldrich. Oxidized alginate was synthesized according to literature methods. See, for example, K. H. Bouhadir, et al., Degradation of partially oxidized alginate and its potential application for tissue engineering, 17 BIOTECHNOL. PROG. 945 (2001), incorporated by reference herein in its entirety. Alginate was dissolved in deionized water. Subsequently, sodium meta-periodate (Sigma-Aldrich) was added to the solution, which was stirred in the dark for 6 hours. The periodate was quenched with ethylene glycol (VWR Chemicals). Ethanol and sodium chloride were added to the mixture so as to precipitate the oxidized alginate product. The precipitate oxidized alginate product was vacuum filtered and washed with ethanol. The filtered product was lyophilized for one week to remove trace solvent. The oxidized alginate product was in the form of a white powder.

### 2. Click Alginate.

Click alginate was kindly provided by Dr. Alexander Stafford, from the David J. Mooney Research Group. The alginate oxidation degree was 5%, and the norbornene-tetrazine functionalization degree was 250 equivalents of functional groups per polymer strand.

### 3. Purified Human Immunoglobulin (IgG).

Purified human Immunoglobulin (IgG) was purchased from Equitech-Bio. The lyophilized powder was dialyzed against sodium acetate buffer, 10 mM, pH 5.5 in a dialysis bag with a molecular weight cutoff of 20,000 for 48 hours. The solvent was changed three times in regular time intervals.

### 4. Acoustophoretic Printer.

The wave generator of the acoustophoretic printer produced a sinusoidal signal, and the voltage of the wave generator was increased using an amplifier. The sinusoidal signal was tested at the amplifier output using a current transformer connected to an oscilloscope. The acoustic field was generated in the acoustic chamber with a system transducer-emitter.

The fluid to be printed was injected with a syringe pump into a glass nozzle, which was placed in a hollow cylinder referred to as the sub-wave. To decrease nozzle wettability, the glass was coated with FUSSO sealant. An air flow parallel to the nozzle tip was blown using compressed air, and the pressure of the airflow could be monitored and tuned using an air regulator. The droplets exiting the sub-wave were imaged using a high-speed camera. Below the sub-wave, the collection bath was placed, and optionally, the MIST-In-flight-Crosslinking (“MISTIC”) device.

### 5. MISTIC Device.

The MISTIC device included a 3D printed cone, opened at the top and bottom. The crosslinking aerosol was generated using a commercial air moisturizer, and was pumped into the MISTIC device through an opening at the top of the cone. After flowing to the bottom of the device, the aerosol was recollected through openings connected to a vacuum line. Below the MISTIC device, the collection bath was placed in a plastic petri dish.

### 6. Collection Baths

In all collection baths, water was used as a solvent. Calcium chloride was used as an ionic crosslinker and added in different amounts, ranging from 0.1% to 10% w/v. To reduce surface tension, Tween 20 (Hardy Diagnostics) was added to the bath, at a concentration of 1 mM. The concentration of 1 mM was reported to lead to a surface tension around 40 mN/m. See, for example, Noor Rehman, et al., Surface and thermodynamic study of micellization of non ionic surfactant/diblock copolymer system as revealed by surface tension and conductivity, 8 J. MATERIALS & ENVIRONMENTAL SCIS. 1161 (2017), incorporated by reference herein in its entirety. To avoid pH fluctuations, the bath was buffered with a 10 mM sodium acetate buffer at pH 5.5. The buffer was obtained by adding sodium acetate trihydrate (Sigma-Aldrich) and acetic acid (MP Chemicals) at the desired ratio.

### B. Printing Protocols

### 1. Alginate and Immunoglobulin.

To print crosslinked alginate hydrogel microparticles with antibody cargo, the following protocol was followed:
a. Sodium acetate buffer (10 mM) was prepared in water.
b. Immunoglobulin G (IgG) was dissolved in 10 mM acetate buffer. The solution was centrifuged at 9,000 Relative Centrifugal Force (rcf) for 5 minutes, then mixed with a pipette. The step was repeated until all of the IgG powder was dissolved in the acetate buffer; usually 2-3 repetitions of the step were required. The IgG solution was loaded into a dialysis cassette (MWCO 20,000, Thermo-Fisher), and dialyzed against 10 mM sodium acetate buffer for 48 hours. The buffer solution was changed 3 times in regular time intervals. After dialysis, the IgG solution was transferred into an Eppendorf tube, centrifuged to remove bubbles, and loaded into a glass syringe.
c. Alginate was dissolved into 10 mM acetate buffer and speed-mixed for about 30 minutes at the desired concentration, which spanned between 2.5% and 7.5% w/v. The alginate solution was loaded into a syringe, filtered with a 5-μm silicon filter, centrifuged at 11,000 rcf for 5 minutes to remove air bubbles, and loaded into a glass syringe (Hamilton).
d. Before injecting the materials, the tubing of the acoustophoretic printer was flushed with sodium acetate buffer. The glass syringes were loaded onto two syringe pumps, connected to a mixing nozzle. Ejection was performed with a total flowrate between 5 and 20 μL/min.
e. Air co-flow was used to eject small droplets using the placement of the nozzle and to avoid clogging the sub-wave. The nozzle was placed at the ejection position, and then the acoustic field was turned on.
f. The MISTIC process was started. The MISTIC device was placed below the sub-wave. The moisturizer generating the aerosol was turned on, and at the same time the vacuum collecting excess aerosol was opened.
g. The petri dish containing the collection bath was placed under the MISTIC device.
h. Pictures of the droplets were taken on a microscope (Olympus IX71).

### 2. Oxidized Alginate and Click Oxidized Alginate

To print crosslinked alginate hydrogel microparticles with antibody cargo, the following protocol was followed:
a. Oxidized alginate or click oxidized alginate was dissolved into water and speed-mixed for about 30 minutes at a concentration of 5% w/v. The alginate solution was filtered with a 5-μm silicon filter, centrifuged at 11,000 rcf for 5 minutes to remove air bubbles, and loaded into a glass syringe (Hamilton). The hydrogel precursors were always freshly prepared due to the tendency for oxidized alginate to undergo hydrolysis in water.
b. Before injecting the materials, the tubing of the acoustophoretic printer was flushed with water. To manufacture oxidized alginate microparticles, a single syringe was connected to the nozzle inlet. Alternatively, to manufacture click oxidized alginate microparticles, two syringes were connected to a mixing nozzle, in order to mix the norbornene and tetrazine chemistries right before ejection. The total flow rate of the syringe pump was set between 5 and 20 μL/min.
c. Air co-flow was used to eject small droplets during the placement of the nozzle and to avoid clogging the sub-wave. The nozzle was placed at the ejection position, and then the acoustic field was turned on.
d. The petri dish containing the collection bath was placed under the sub-wave.
e. Pictures of the droplets were taken on a microscope (Olympus IX71).

### C. Results and Discussion

### 1. Particle Shape and Diameter.

In order to understand the formation of defects, a closer look was taken on the impact of the crosslinked hydrogel microparticles on the collection bath surface.

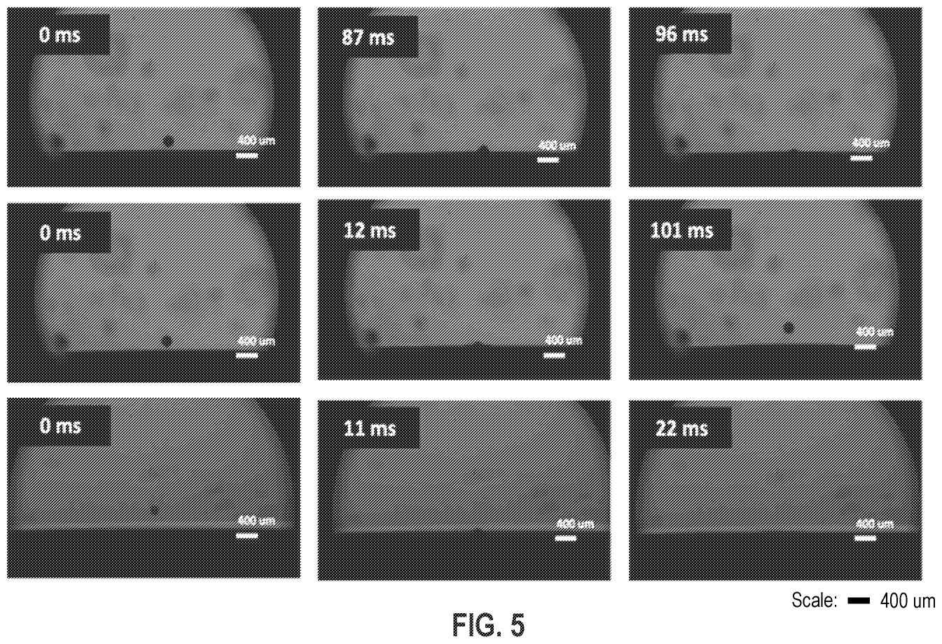

Different values of the dimensional Weber particle number (We) represent different impact events. See, for example, Kuo-Long Pan, et al., Binary droplet collision at high Weber number, 80 PHYSICAL REVIEW E 036301 (2009), incorporated by reference herein in its entirety. If solution density, surface tension, and droplet diameter are fixed, the We number increases for increasing particle velocities. For low We numbers, there is static coalescence, which means that the droplet impacts gently on the liquid surface of the collection bath, as illustrated in the top row of images in FIG. 5. After depositing on the surface of the collection bath, the droplet slowly sinks into the collection bath. For higher We numbers, the droplet compresses the air cushion above the collection bath, without displacing the collection bath, as illustrated in the middle row of images in FIG. 5. When the air cushion decompresses, the droplet was pushed upwards, consequently bouncing on the collection bath surface. Falling down again by gravity, the droplet is significantly slowed down and undergoes static coalescence. For high We numbers, the droplet quickly merges with the collection bath, which is a phenomenon named dynamic coalescence, as illustrated in the bottom row of images in FIG. 5. For producing spherical droplets, dynamic coalescence is preferred, because the droplet is quickly surrounded by crosslinker solution, avoiding formation of a tail defect.

To avoid static coalescence or bouncing, the We number must be kept high. For a decreasing droplet diameter, a high Weber number may be achieved by increasing droplet velocity. When the droplet is ejected by acoustic force only, the droplet reaches a terminal velocity during its fall. For water droplets falling through stagnant air, this velocity is proportional to the droplet size. See, for example, R. Gunn & G. D. Kinzer, The Terminal Velocity of Fall for Water Droplets in Stagnant Air, 6 J. METEOROL. 243 (1949), incorporated by reference herein in its entirety. The smaller the droplet, the lower the terminal velocity, and the higher the probability of defects. An air co-flow parallel to the nozzle may be introduced to the speed up the droplet during flight. However, such an air co-flow would recirculate on the collection bath surface, slowing down the droplets and increasing the probability of the droplet bouncing.

Another parameter affecting particle shape may be the amount of biological cargo contained in the droplet. Because the interactions between the polymer and the crosslinking agent and between polymer and biological cargo are electrostatic, there is competition among crosslinking agent ions and biological cargo for the negative charges on the polymer. The competition causes the polymer matrix to crosslink at a slower rate and results in less of an opportunity to retain the spherical droplets.

Because defects in the droplets were generated upon impact with the collection bath surface, and because adding more antibody cargo would make defect generation easier, a way to ensure spherical droplet shape regardless of droplet size would be to crosslink the droplets during flight. To achieve droplet crosslinking during flight, a process was designed whereby particles fall and travel through an aerosol containing crosslinking agent solution. The impacts between the aerosol containing crosslinking agent solution droplets and polymer droplets cause the shell of the precursor solution to gel. By crosslinking during flight, regardless of droplet size and velocity, the shape of the microparticle shell was fixed as spherical before impacting the collection bath.

The process whereby droplet crosslinking is achieved during flight, known as MISTIC, begins with acoustophoretic printing of a precursor solution including a polymer and antibodies in buffered aqueous solution. After detachment of a droplet of the solution, the precursor droplet falls through the MISTIC device, in which the droplet impacts aerosol droplets including high crosslinking agent concentration (for example, 10% w/v CaCl₂)). As the crosslinked microparticles land in the collection bath, the microparticles are collected in a buffered water solution including crosslinking agent dissolved in the water. The presence of crosslinking agent after collection ensures that the crosslinked microparticles do not dissolve, while the acidic buffer ensures an electrostatic interaction between the polymer and the biological cargo.

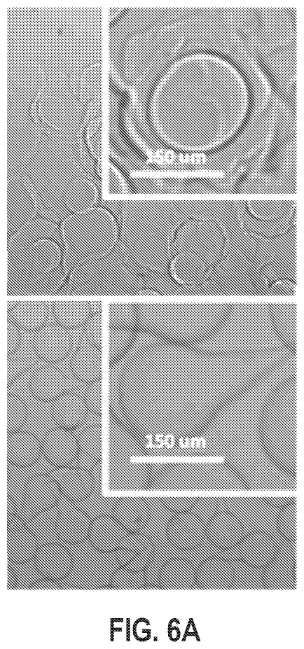

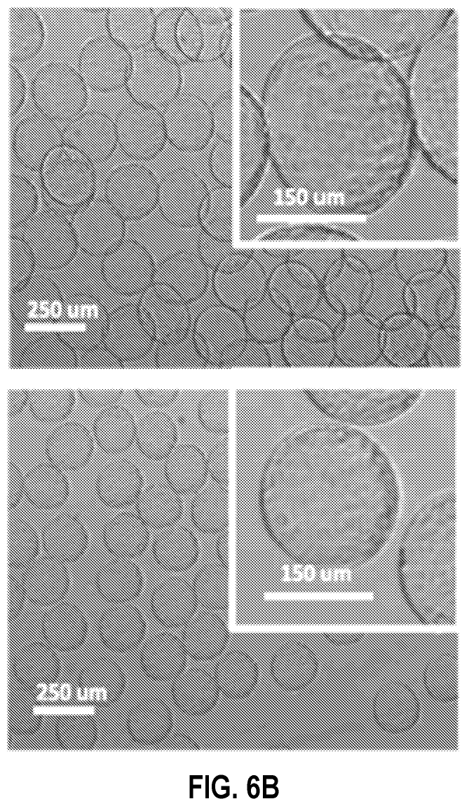

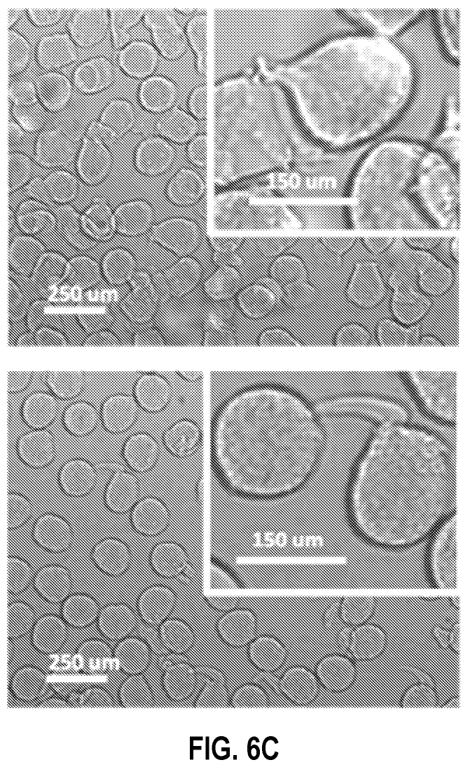

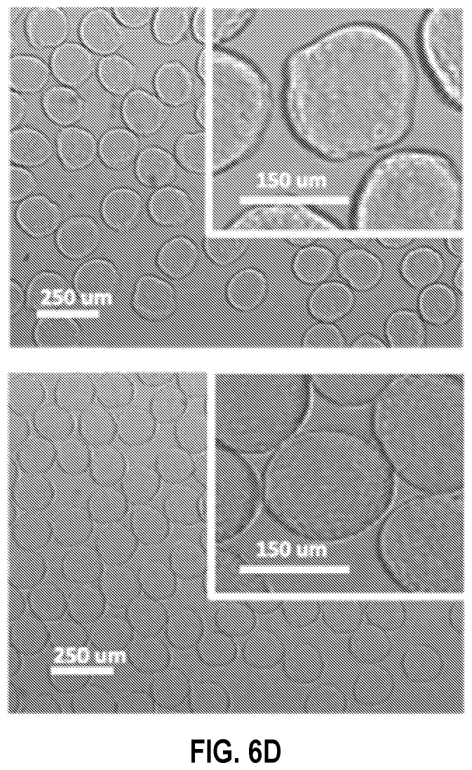

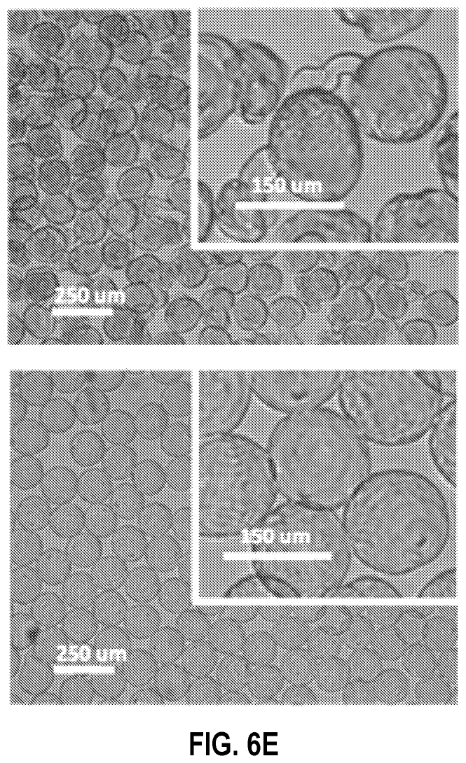

The preliminary experiments to analyze crosslinking during droplet flight were carried out without biological cargo, as illustrated in FIGS. 6A, 6B, 6C, 6D, and 6E. Alginate particles were printed without the MISTIC procedure, and without any surfactant in the collection bath. Two different crosslinking agent concentrations (0.1% w/v (top) and 1% w/v (bottom) aqueous calcium chloride) were tested in all experiments. For both crosslinking agent concentrations, particles produced without MISTIC presented significant shape defects, such as amorphous clumps of gel, or particles with a large tail, as illustrated in FIG. 6A. For the microparticles illustrated in FIG. 6A, the alginate concentration was 5% w/v, and the surface tension was 70 mN/m. Keeping all parameters fixed while adding the MISTIC procedure improved the shape of the microparticles. With the MISTIC procedure, the particles were spherical for both crosslinking agent concentrations, as illustrated in FIG. 6B. For the microparticles illustrated in FIG. 6B, the alginate concentration was 5% w/v and the surface tension was 70 mN/m.

Decreasing the polymer concentration in the alginate precursor solution was necessary to ensure higher biological cargo concentration in a droplet when using a mixing nozzle, because the concentration in the alginate precursor solution was limited and had a maximal concentration of from about 7.5% to about 10% w/v. Lowering the flow rate fraction from the mixing nozzle allowed for a higher concentration of biological cargo concentration in the precursor solution. When the polymer concentration was decreased, less crosslinking sites were available, so the gelation rate of the shell was slower, which was confirmed by the appearance of tail defects when the polymer concentration was halved to 2.5% w/v, as illustrated in FIG. 6C. For the microparticles illustrated in FIG. 6C, the surface tension remained 70 mN/m.

An increase in impacts between the hydrogel precursor droplets and the aerosol droplets including crosslinking agent would provide more crosslinking and a more spherical particle shape. Likewise, reducing the surface tension of the collection bath to 40 mN/m would result in a more spherical particle shape. The surface tension of the collection bath was accomplished by adding Tween 20, a surfactant that is FDA-approved for various biomedical applications, including intravenous injections. The addition of Tween 20 improved the wetting of the hydrogel precursor droplet upon impact, and the microparticles produced were spherical, as illustrated in FIG. 6D. For the microparticles illustrated in FIG. 6D, the polymer concentration remained 2.5% w/v.

When adding biological cargo, such as a concentration of 100 mg/mL, there is competition for the negative charges on the polymer between the calcium ions and the positively charged biological cargo. The competition for the negative charges on the polymer was proven by the appearance of defects for the lowest salt concentration conditions after adding IgG cargo, while no defects were observable for the 1% crosslinking agent concentration, as illustrated in FIG. 6E.

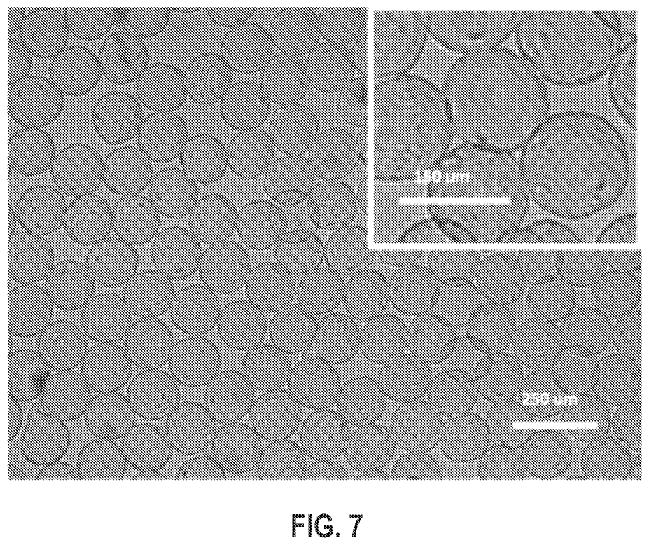

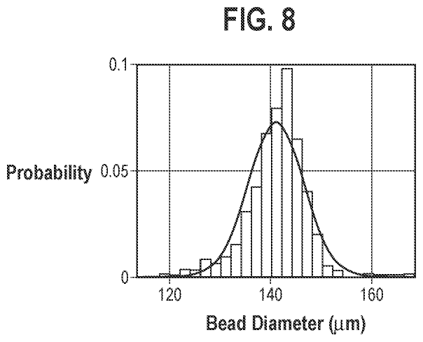

Implementing the MISTIC procedure and lowering the surface tension allowed the production, for the first time, of alginate microparticles with 2.5% w/v polymer concentration, an antibody concentration of 100 mg/mL, a spherical shape as illustrated in FIG. 7, and an average diameter of 141 μm, as illustrated in FIG. 8. The coefficient of variation of 3.9% for the particle diameter distribution was evidence of high monodispersity. By using MISTIC, the average particle diameter was reduced from above 380 μm to below 150 μm, which corresponded to a diameter decrease of about 60% and a volume decrease of about 95%. It is expected that a further decrease of 60% in diameter (an average diameter of about 60 μm) would allow the granular gel to flow in a conventional subcutaneous injection needle, thus making the formulation injectable.

### 2. Polymer Matrix Clearability.

To deliver their biological cargo, the hydrogel microparticles should be injected into the subcutaneous space. Alginate is stable in water, and the human body does not have the enzymes required to degrade alginate. Consequently, after injection, the alginate beads would simply accumulate under the skin rather than degrade and release the biological cargo.

However, oxidized alginate does undergo hydrolytic degradation. See, for example, C. G. Gomez, et al., Oxidation of sodium alginate and characterization of the oxidized derivatives, 67 CARBOHYDR. POLYM. 296 (2007), incorporated by reference herein in its entirety. The threshold for clearance of a polymer in the human body is a molecular weight below 50 kDa, and oxidized alginate has been demonstrated to drop below 50 kDa in solutions at both neutral and basic pH values. See, for example, K. H. Bouhadir, et al. (2001). Oxidized alginate has also been tested in vivo, which demonstrated oxidized alginate to be an inert material that caused no immunogenic reactions in the subcutaneous space. See, for example, R. M. Desai, et al., Versatile click alginate hydrogels crosslinked via tetrazine-norbornene chemistry, 50 BIOMATERIALS 30 (2015), incorporated by reference herein in its entirety.

Alginate is a polysaccharide composed of mannuronate (M) and guluronate (G) blocks, mannuronate and guluronate being two conformations of the same monosaccharide, as shown in the structure of alginate below.

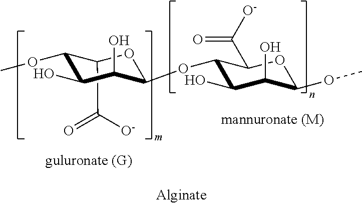

The G blocks of alginate are the blocks responsible for the egg-shell structure during crosslinking. The oxidizing agent adding during synthesis of oxidized alginate reacts selectively with the G blocks of alginate, decreasing the number of available crosslinking sites. Therefore, the gelation properties of the precursor solution might be compromised after oxidation of the alginate.

Because the oxidation of alginate affects the material properties of the alginate polymer, the oxidation reaction was performed for five different amounts of oxidizing agent relative to amounts of alginate. The molecular weight of the oxidized alginate decreased for an increasing amount of oxidizing agent, but the molecular weight of the oxidized alginate reached a plateau above 0.3 equivalents of oxidizing agent. Accordingly, the optimum amount of oxidizing agent for the oxidation of alginate was determined to be between 0 and 0.3 equivalents of oxidizing agent relative to alginate precursor.

Because viscosity is directly correlated with molecular weight, the viscosity of the oxidized alginates decreased by up to two orders of magnitude relative to non-oxidized alginates. The decrease in viscosity of the oxidized alginates is beneficial, because the polymer concentration in the precursor solution can be increased before reaching a critical viscosity that would generate high pressure losses in an acoustophoretic printer tubing.

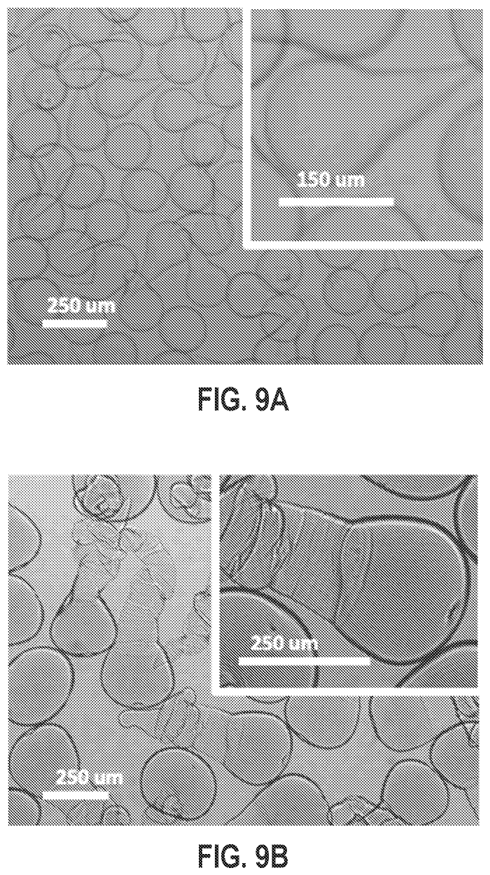

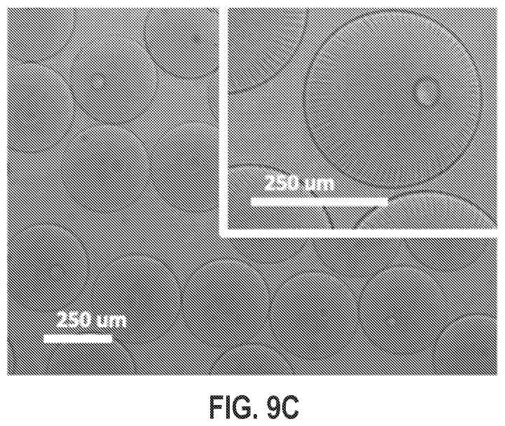

Compared to non-oxidized alginate, as illustrated in FIG. 9A, oxidized alginate precursor at the same precursor concentration (alginate at 5% w/v) and with the same collection bath composition (1% w/v CaCl₂) with a surface tension of 70 mN/m) forms particles with larger tail defects, as illustrated in FIG. 9B, confirming that the oxidation reaction compromises the gelation of the polymer matrix. By increasing the crosslinking agent concentration by one order of magnitude (10% w/v CaCl₂)), the concentration of crosslinking agent cations compensated for the lower density of crosslinking sites, and spherical particles were formed, as illustrated in FIG. 9C, indicating that forming spherical oxidized alginate microparticles is possible if the crosslinking rate during droplet flight or in the collection bath is increased.

Oxidized alginate is clearable, but the tunability of the degradation rate of the oxidized alginate is limited. At neutral pH, the polymer strands degrade to 50 kDa in approximately two weeks. See, for example, K. H. Bouhadir, et al. (2001). If a slower degradation is desired, the tuning of the degree of oxidation of alginate would be difficult, because very low equivalent amounts of oxidizing agent would be required, and the amount of G blocks in alginate is subject to natural batch-to-batch variations.

“Click” oxidized alginate is an oxidized alginate that has been functionalized with norbornene and tetrazine groups, which can react by reverse Diels-Alder as shown below to form covalent bonds among alginate strands. By tuning the amount of functionalization, the density of covalent crosslinks, and consequently, the degradation rate, may be regulated.

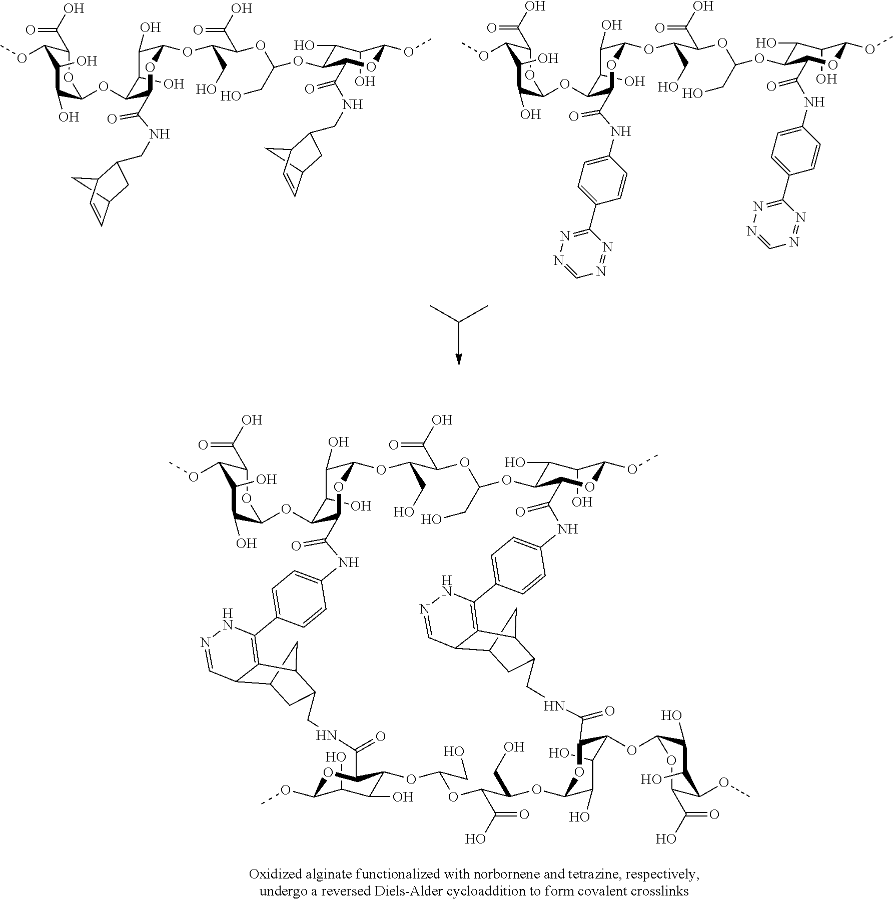

The “click” alginate in this example required approximately 30 minutes to react, which means that conventional microparticle production methods would require an emulsion in order to form the microparticles. There are numerous examples of “click” chemistry “pairs,” known in the art to address and optimize different rates of reactivity, reaction conditions, and stability. See, for example, Z. Geng, et al., Click chemistry strategies for the accelerated synthesis of functional macromolecules, 59 J. POLYMER SCI. 963 (2021); Y. Takayama, et al., Click Chemistry as a tool for cell engineering and drug delivery, 24 MOLECULES 172 (2019); each of which is incorporated herein by reference in its entirety.

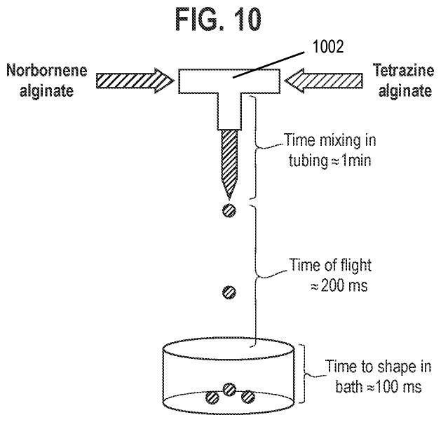

Referring to FIG. 10, because the precursor solutions of alginate functionalized with norbornene and with tetrazine could not be mixed before droplet printing, a mixing nozzle 1002 was used to mix the precursor alginate solutions. There was a brief time window in which the covalent crosslinking could occur. After leaving the mixing nozzle 1002, the two precursor alginate solutions flowed in the mixing nozzle 1002 with a residence time of about 1 minute. After ejection, the time of flight from the mixing nozzle 1002 to the collection bath surface was about 200 milliseconds (ms). In the bath, the particles retained the spherical shape for a brief period (less than about 100 ms), before the alginate would start diffusing into the water. Because the precursor solutions could not be gelled in the nozzle, the only time window available for gelling the precursor solutions was during droplet flight. A faster intermediate type of gelation was required to retain the spherical particle shape.

The faster immediate type of crosslinking was ionic, because the click functionalization did not hinder the formation of the polymer-calcium egg-shell structure. The particles landed in a calcium chloride collection bath, where they crosslinked instantly, fixing the spherical shape immediately. By letting the microparticles sit in the salt collection bath for 2 hours, the click reaction happened and the particles crosslinked covalently as well. The salt was removed with a washing step using Tris buffer, leaving particles linked by covalent bonds only.

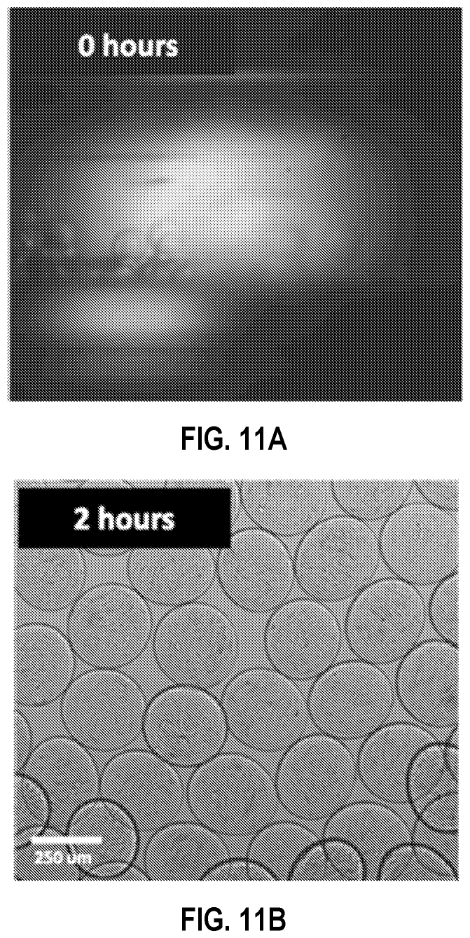

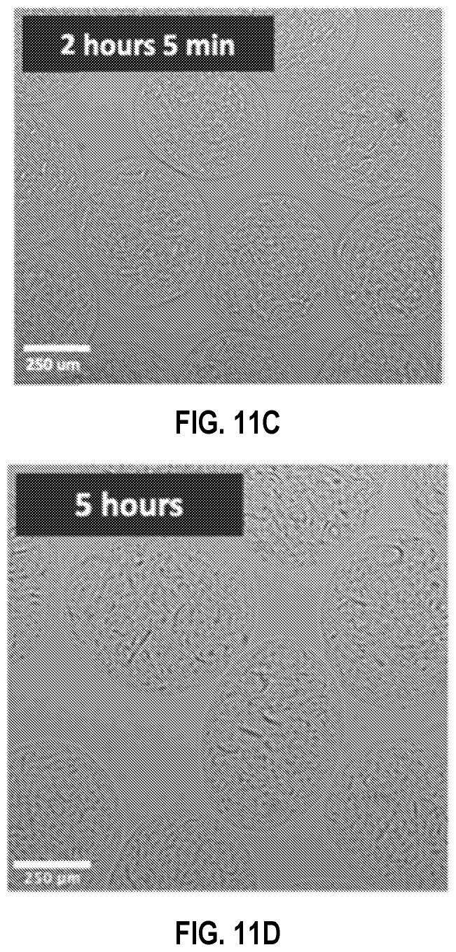

The ionic crosslinking protocol was experimentally validated. The particles landed in a calcium chloride collection bath, as illustrated in FIG. 11A, where they had the same aspect ratio as non-oxidized alginate particles. The particles linked covalently as well by 2 hours, as illustrated in FIG. 11B. Immediately after removing the salt by washing with buffer, five minutes after particle resuspension, the particles changed appearance and swelled, as illustrated in FIG. 11C. After waiting three hours, the particles retained the same appearance and dimension, as illustrated in FIG. 11D, suggesting that most of the ionic crosslinks were removed within minutes after the washing step.

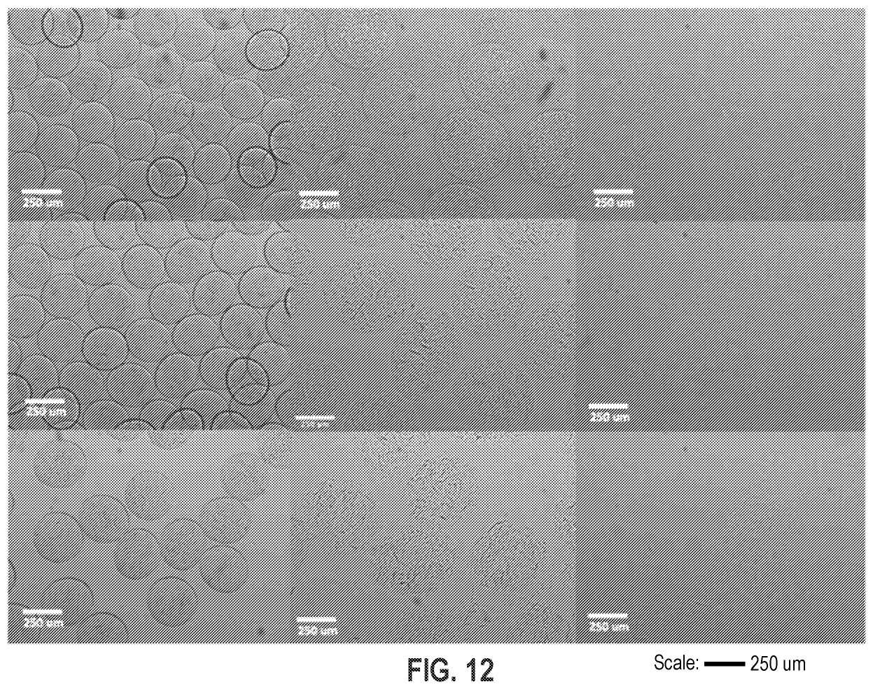

It was hypothesized that the amount of salt in the collection bath may have affected the swelling behavior of the particles, as well as the degradation rate of the particles. Therefore, an experiment monitoring swelling and degradation of the microparticles for different initial concentrations of calcium chloride, as illustrated in FIG. 12, for 0.1% w/v (bottom row), 1.0% w/v (middle row), and 10% w/v (top row), over a period of three weeks was performed. The initial appearance of the particles was identical for the three different orders of magnitude of concentrations of calcium (left column). The swelling behavior (middle column, at 3 hours) was also identical in all cases (9-fold average volume increase), with the only exception being the 10% w/v calcium case, for which the particles welled less than the other two concentrations (7-fold average volume increase). After three weeks (right column), the particles of all three samples degraded hydrolytically.

### D. Conclusion

The formation of particle defects was analyzed and clarified by introducing the Weber number. The particle production process was modified based on the insights gained on defects formation, and a new type of crosslinking process, the MISTIC, was introduced to overcome physical limitations of the acoustophoretic printer. The principle of MISTIC was validated with experiments, and together with the use of surfactant, the hypotheses of the effects of polymer concentration, crosslinking agent concentration, and antibody concentration on particle shape were confirmed. Conditions leading to spherical alginate particles with antibody cargo and with a 60% diameter reduction were discovered. The size distribution of the hydrogel microparticles was analyzed, and evidence of monodispersity (CV<4%) was found. The spherical shape of the particles ensured reproducible rheological properties and a robust quantification of particle amounts and drug dosage in the delivery platform. The smaller diameter approaches the size required by standard subcutaneous injection nozzles.

To make alginate clearable, the material was oxidized following literature protocols. By screening over different amounts of oxidizing agent, the effect of oxidation on molecular weight and viscosity was investigated. The gelation properties of oxidized alginate were tested, and the particle shape confirmed that oxidation slowed down the crosslinking rate. Spherical particles were obtained for high concentrations of crosslinking agent, demonstrating the possibility to gel the particles completely. To allow for a better tunability of the degradation rate, covalent crosslinks were added by introducing click chemistry functionalization. The microparticle production protocol was adjusted to overcome the limitation of the long covalent crosslinking time, and validated experimentally.

Particle formation, swelling, and degradation were retained for different initial crosslinker concentrations, demonstrating that the degree of covalent crosslinking was independent of the degree of ionic crosslinking. Accordingly, the hydrogel matrix is degradable in a physiological environment, and would not accumulate in the subcutaneous space after repeated injection. Further, the rate of degradation could be decreased, for applications in which a slower particle clearance would be useful, such as tumor embolization.

Although the present disclosure has been described with reference to examples and the accompanying drawings, the present disclosure is not limited thereto, but may be variously modified and altered by those skilled in the art to which the present disclosure pertains without departing from the spirit and scope of the present disclosure.

The subject-matter of the disclosure may also relate, among others, to the following aspects:

A first aspect relates to a method of preparing a particle, comprising: releasing a liquid droplet from a droplet generator into a first fluid comprising an aerosol comprising a crosslinking agent, the droplet comprising a formulation comprising a polymer; contacting the liquid droplet with the crosslinking agent in the first fluid; crosslinking the liquid droplet to form the particle comprising a crosslinked matrix; and collecting the particle.

A second aspect relates to the method of aspect 1, wherein the particle is a gelled particle comprising a crosslinked hydrogel matrix, and the polymer is a hydrogel precursor.

A third aspect relates to the method of any preceding aspect, wherein the formulation comprises a cargo, and the cargo is dispersed in the crosslinked matrix.

A fourth aspect relates to the method of aspect 3, wherein the cargo is a biologic.

A fifth aspect relates to the method of any preceding aspect, further comprising: generating an acoustic field in the first fluid with an oscillating emitter; and detaching the droplet from the droplet generator by acoustic forces from the acoustic field.

A sixth aspect relates to the method of any preceding aspect, comprising releasing a continuous stream of the liquid droplets from the droplet generator for a period of time.

A seventh aspect relates to the method of any preceding aspect, wherein the aerosol comprises an aqueous solution of the crosslinking agent.

An eighth aspect relates to the method of any preceding aspect, wherein the first fluid is air.

A ninth aspect relates to the method of any preceding aspect, further comprising flowing air with the liquid droplet during the releasing.

A tenth aspect relates to the method of any preceding aspect, wherein a velocity of the aerosol is the same as a velocity of the liquid droplet.

An eleventh aspect relates to the method of any preceding aspect, wherein the crosslinking comprises forming a crosslinked shell prior to the collecting.

A twelfth aspect relates to the method of aspect 11, wherein the crosslinked shell is formed around the cargo.

A thirteenth aspect relates to the method of any preceding aspect, wherein the contacting, crosslinking, and/or the collecting are in the absence of ultraviolet radiation.

A fourteenth aspect relates to the method of any preceding aspect, wherein the collecting is in a collection bath.

A fifteenth aspect relates to the method of aspect 14, wherein the collection bath comprises a solution of the crosslinking agent.

A sixteenth aspect relates to the method of aspect 15, wherein the solution comprises an acidic buffer.

A seventeenth aspect relates to the method of any preceding aspect, wherein the particle has an average diameter of less than 250 μm.

An eighteenth aspect relates to the method of aspect 17, wherein the average diameter is less than 150 μm.

A nineteenth aspect relates to the method of aspect 18, wherein the average diameter is less than 100 μm.

A twentieth aspect relates to the method of aspects 17-19, wherein the average diameter has a coefficient of variation of less than about 5.0%.

A twenty-first aspect relates to the method of aspect 20, wherein the coefficient of variation is less than about 4.0%.

A twenty-second aspect relates to the method of any preceding aspect, wherein the particle has a sphericity of greater than about 0.6.

A twenty-third aspect relates to the method of aspects 3-22, wherein a concentration of the cargo in the particle is from about 50 mg/mL to about 500 mg/mL.

A twenty-fourth aspect relates to the method of any preceding aspect, wherein the formulation has a viscosity in a range of from 100 mPa·s to about 500,000 mPa·s.

A twenty-fifth aspect relates to the method of any preceding aspect, wherein the crosslinking agent is calcium chloride.

A twenty-sixth aspect relates to the method of aspects 7-25, wherein a concentration of the crosslinking agent in the solution is from about 0.1% w/v to about 10% w/v.

A twenty-seventh aspect relates to the method of aspects 3-26, wherein the cargo is homogeneously dispersed in the crosslinked matrix.

A twenty-eighth aspect relates to the method of aspects 3-27, wherein the particle comprises a polymer-to-cargo mass:mass ratio in a range of from about 10:1 to about 1:1000.

A twenty-ninth aspect relates to the method of aspects 3-28, wherein the formulation comprises the cargo at a concentration of at least about 20 mg/mL and/or as high as about 1000 mg/mL.

A thirtieth aspect relates to the method of aspects 4-29, wherein the formulation has a pH below an isoelectric point of the biologic.

A thirty-first aspect relates to the method of aspects 4-29, wherein the formulation has a pH above an isoelectric point of the biologic.

A thirty-second aspect relates to the method of any preceding aspect, wherein the formulation further comprises an excipient selected from the group consisting of a buffering agent, an amino acid, an antioxidant, a surfactant, a preservative, and mixtures thereof.

A thirty-third aspect relates to the method of any preceding aspect, wherein the formulation further comprises amorphous aluminum hydroxyphosphate sulfate (AAHS), aluminum hydroxide, aluminum phosphate, potassium aluminum sulfate, and/or cytosine phosphoguanine (CpG).

A thirty-fourth aspect relates to the method of aspects 14-33, wherein a surface tension of the collection bath is from about 15 mN/m to about 80 mN/m.

A thirty-fifth aspect relates to the method of any preceding aspect, further comprising washing the particle to remove the crosslinking agent from the particle after a predetermined amount of time.

A thirty-sixth aspect relates to the method of aspects 4-35, wherein the biologic comprises a protein, a checkpoint inhibitor, a hormone, a peptide, a nucleic acid, a mammalian cell, a micro-organism, a small molecule, a bacterium, a drug, a cytokine, insulin, Botulinum toxin type A, Botulinum toxin type B, bovine serum albumin (BSA), human immunoglobulin G (IgG), Fc fusion protein, an anticoagulant, a blood factor, a bone morphogenetic protein, an engineered protein scaffold, an enzyme, and/or a thrombolytic.

A thirty-seventh aspect relates to the method of aspects 4-36, wherein the biologic is a monoclonal antibody, an antibody-drug conjugate, a bispecific antibody, an interleukin, an interferon, a tumor necrosis factor, a chemokine, and/or a growth factor.

A thirty-eighth aspect relates to the method of any preceding aspect, wherein a route of administration of the particle into a human body is selected from the group consisting of: uricular, buccal, conjunctival, cutaneous, dental, electro-osmotical, endocervical, endosinusial, endotracheal, enteral, epidural, extra-amniotical, extracorporeal, infiltration, inhalation, interstitial, intra-abdominal, intra-amniotical, intra-arterial, intra-articular, intrabiliary, intrabronchial, intrabursal, intracardial, intracartilaginous, intracaudal, intracavitary, intracerebral, intracisternal, intracorneal, intracoronal, intracoronary, intracorporus cavernosum, intradermal, intradiscal, intraductal, intraduodenal, intradural, intraepidermal, intraesophageal, intragastrical, intragingival, intraileal, intralesional, intraluminal, intralymphatical, intramedullar, intrameningeal, intramuscular, intraocular, intraovarian, intrapericardial, intraperitoneal, intrapleural, intraprostatical, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nastrogastrical, occlusive dressing technique, ophthalmical, oral, oropharyngeal, parenteral, percutaneous, periarticular, peridural, perineural, periodontal, rectal, retrobulbar, soft tissue, subarachnoidial, subconjunctival, subcutaneous, sublingual, submucosal, topical, transdermal, transmucosal, transplacental, transtracheal, transtympanic, ureteral, urethral, and/or vaginal.

A thirty-ninth aspect relates to the method of any preceding aspect, wherein the formulation comprises the polymer at a concentration of at least about 20 mg/mL and/or as high as about 1000 mg/mL; and/or wherein the polymer comprises an acrylate precursor, an acrylonitrile precursor, an alginate precursor, an agar precursor, an agarose precursor, a carboxymethylcellulose precursor, a carrageenan precursor, a chitosan precursor, a chondroitin sulfate precursor, a collagen precursor, a dextran precursor, a fibrin precursor, a gelatin precursor, a hydroxyethylcellulose precursor, a hyaluronate precursor, a xanthan precursor, a polylysine precursor, a poly(acrylic) acid precursor, a precursor for poly(ethylene glycol) and/or a derivative thereof, a precursor for cellulose and/or a derivative thereof, a precursor for poly(propylene glycol) and/or a derivative thereof, a precursor for polylactide and/or a derivative thereof, a precursor for poly(glycolic acid) and/or a derivative thereof, a precursor for poly(propylene fumarate) and/or a derivative thereof, a precursor for polycaprolactone and/or a derivative thereof, a precursor for polyhydroxybutyrate and/or a derivative thereof, a precursor for polyacrylates and/or derivatives thereof, a precursor for poly(vinylpyrrolidone) and/or a derivative thereof, and/or a precursor for poly(ethylenimine) and/or a derivative thereof.

A fortieth aspect relates to the method of aspect 39, wherein a first portion of the polymer precursor comprises a first moiety and a second portion of the polymer precursor comprises a second moiety; wherein the first moiety undergoes a click chemistry reaction with the second moiety; and wherein the first portion and the second portion are mixed in the droplet generator.

A forty-first aspect relates to the method of aspect 40, wherein the first moiety is a norbornenyl group and the second moiety is a tetrazinyl group.

A forty-second aspect relates to the method of any preceding aspect, wherein the droplet generator comprises a mixing nozzle.

A forty-third aspect relates to a composition, comprising: a particle formed from a liquid droplet comprising a formulation comprising a polymer, the particle comprising a crosslinked matrix; wherein the particle comprises a particle surface comprising a plurality of surface pores, the plurality of surface pores having an average valley depth and/or an average diameter of from about 0.1 μm to about 20 μm.

A forty-fourth aspect relates to the composition of aspect 43, wherein the particle is a gelled particle comprising a crosslinked hydrogel matrix, and the polymer is a hydrogel precursor.

A forty-fifth aspect relates to the composition of aspects 43-44, wherein the formulation comprises a cargo, and the cargo is dispersed in the crosslinked matrix.

A forty-sixth aspect relates to the composition of aspects 43-45, wherein the cargo is a biologic.

A forty-seventh aspect relates to the composition of aspects 43-46, wherein the particle has an average diameter of less than 250 μm.

A forty-eighth aspect relates to the composition of aspects 43-47, wherein the particle has a sphericity of greater than about 0.6.

A forty-ninth aspect relates to the composition aspects 47-48, wherein the average diameter of the particle has a coefficient of variation of less than about 5.0%.

A fiftieth aspect relates to the composition of aspect 49, wherein the coefficient of variation is less than about 4.0%.

A fifty-first aspect relates to the composition of aspects 45-50, wherein a concentration of the cargo in the particle is from about 50 mg/mL to about 500 mg/mL.

A fifty-second aspect relates to the composition of aspects 43-51, wherein the formulation has a viscosity in a range of from about 100 mPa·s to about 500,000 mPa·s.

A fifty-third aspect relates to the composition of aspects 43-52, wherein the composition is acoustophoretically printed.

A fifty-fourth aspect relates to the composition of aspects 43-53, wherein the crosslinked matrix comprises acrylate, acrylonitrile, alginate, agar, agarose, carboxymethylcellulose, carrageenan, chitosan, chondroitin sulfate, collagen, dextran, fibrin, gelatin, hyaluronate, hydroxyethylcellulose, xanthan, polylysine, poly(acrylic) acid, poly(ethylene glycol) and derivatives thereof, cellulose and derivatives thereof, poly(propylene glycol) and derivatives thereof, polylactide and derivatives thereof, poly(glycolic acid) and derivatives thereof, poly(propylene fumarate) and derivatives thereof, polycaprolactone and derivatives thereof, polyhydroxybutyrate and derivatives thereof, polyacrylates and derivatives thereof, poly(vinylpyrrolidone) and derivatives thereof, and/or poly(ethylenimine) and derivatives thereof.

A fifty-fifth aspect relates to the composition of aspects 43-54, wherein the polymer comprises an acrylate precursor, an acrylonitrile precursor, an alginate precursor, an agar precursor, an agarose precursor, a carboxymethylcellulose precursor, a carrageenan precursor, a chitosan precursor, a chondroitin sulfate precursor, a collagen precursor, a dextran precursor, a fibrin precursor, a gelatin precursor, a hydroxyethylcellulose precursor, a hyaluronate precursor, a xanthan precursor, a polylysine precursor, a poly(acrylic) acid precursor, a precursor for poly(ethylene glycol) and/or a derivative thereof, a precursor for cellulose and/or a derivative thereof, a precursor for poly(propylene glycol) and/or a derivative thereof, a precursor for polylactide and/or a derivative thereof, a precursor for poly(glycolic acid) and/or a derivative thereof, a precursor for poly(propylene fumarate) and/or a derivative thereof, a precursor for polycaprolactone and/or a derivative thereof, a precursor for polyhydroxybutyrate and/or a derivative thereof, a precursor for polyacrylates and/or derivatives thereof, a precursor for poly(vinylpyrrolidone) and/or a derivative thereof, and/or a precursor for poly(ethylenimine) and/or a derivative thereof.

A fifty-sixth aspect relates to the composition of aspects 46-55, wherein the biologic comprises a protein, a checkpoint inhibitor, a hormone, a peptide, a nucleic acid, a mammalian cell, a micro-organism, a small molecule, a bacterium, a drug, a cytokine, insulin, Botulinum toxin type A, Botulinum toxin type B, bovine serum albumin (BSA), human immunoglobulin G (IgG), Fc fusion protein, an anticoagulant, a blood factor, a bone morphogenetic protein, an engineered protein scaffold, an enzyme, and/or a thrombolytic.

A fifty-seventh aspect relates to the composition of aspects 46-56, wherein the biologic is a monoclonal antibody, an antibody-drug conjugate, a bispecific antibody, an interleukin, an interferon, a tumor necrosis factor, a chemokine, and/or a growth factor.

A fifty-eighth aspect relates to the composition of aspects 45-57, wherein the cargo is homogeneously dispersed in the crosslinked matrix.

A fifty-ninth aspect relates to the composition of aspects 45-58, wherein the particle comprises a polymer-to-cargo mass:mass ratio in a range of from about 10:1 to about 1:1000.

A sixtieth aspect relates to the composition of aspects 43-59, further comprising a shell encapsulating the particle, the shell comprising a biocompatible polymer.

A sixty-first aspect relates to the composition of aspect 60, wherein the biocompatible polymer comprises chitosan and derivatives thereof, cationic dextran and derivatives thereof, cationic cellulose and derivatives thereof, cationic gelatin and derivatives thereof, poly(2-N,N-dimethylaminoethylmethacrylate) and derivatives thereof, poly-L-lysine and derivatives thereof, polyethylenimine and derivatives thereof, and/or poly(amidoamine)s and derivatives thereof.

A sixty-second aspect relates to the composition of aspects 47-61, wherein the particle has an average diameter of less than 150 μm.

A sixty-third aspect relates to the composition of aspect 62, wherein the particle has an average diameter of less than 100 μm.

A sixty-fourth aspect relates to the composition of aspects 43-63, wherein the formulation comprises the polymer at a concentration of at least about 10 mg/mL and/or as high as about 1000 mg/mL.

A sixty-fifth aspect relates to the composition of aspects 45-64, wherein the formulation comprises the cargo at a concentration of at least about 20 mg/mL and/or as high as about 1000 mg/mL.

A sixty-sixth aspect relates to the composition of aspects 46-65, wherein the formulation has a pH below an isoelectric point of the biologic.

A sixty-seventh aspect relates to the composition of aspects 46-65, wherein the formulation has a pH above an isoelectric point of the biologic.

A sixty-eighth aspect relates to the composition of aspects 43-67, wherein the formulation further comprises an excipient selected from the group consisting of a buffering agent, an amino acid, an antioxidant, a surfactant, a preservative, and mixtures thereof.

A sixty-ninth aspect relates to the composition of aspects 43-68, wherein the formulation further comprises amorphous aluminum hydroxyphosphate sulfate (AAHS), aluminum hydroxide, aluminum phosphate, potassium aluminum sulfate, and/or cytosine phosphoguanine (CpG).

A seventieth aspect relates to the composition of aspects 43-69, wherein a route of administration of the composition into a human body is selected from the group consisting of: uricular, buccal, conjunctival, cutaneous, dental, electro-osmotical, endocervical, endosinusial, endotracheal, enteral, epidural, extra-amniotical, extracorporeal, infiltration, inhalation, interstitial, intra-abdominal, intra-amniotical, intra-arterial, intra-articular, intrabiliary, intrabronchial, intrabursal, intracardial, intracartilaginous, intracaudal, intracavitary, intracerebral, intracisternal, intracorneal, intracoronal, intracoronary, intracorporus cavernosum, intradermal, intradiscal, intraductal, intraduodenal, intradural, intraepidermal, intraesophageal, intragastrical, intragingival, intraileal, intralesional, intraluminal, intralymphatical, intramedullar, intrameningeal, intramuscular, intraocular, intraovarian, intrapericardial, intraperitoneal, intrapleural, intraprostatical, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastrical, occlusive dressing technique, ophthalmical, oral, oropharyngeal, parenteral, percutaneous, periarticular, peridural, perineural, periodontal, rectal, retrobulbar, soft tissue, subarachnoidial, subconjunctival, subcutaneous, sublingual, submucosal, topical, transdermal, transmucosal, transplacental, transtracheal, transtympanic, ureteral, urethral, and/or vaginal.

A seventy-first aspect relates to an in-flight crosslinking device, comprising: an aerosol generator configured to introduce into the device a fluid comprising an aerosol comprising a crosslinking agent; a droplet generator configured to release into the fluid a liquid droplet comprising a polymer, the liquid droplet contacting the crosslinking agent; and a target configured to receive an at least partially crosslinked particle formed from the liquid droplet.

A seventy-second aspect relates to the device of aspect 71, further comprising a chamber configured to confine the fluid, the chamber comprising an opening through which the liquid droplet is released into the fluid and a second opening through which the target receives the at least partially crosslinked particle.

A seventy-third aspect relates to the device of aspects 71-72, wherein the target comprises a collector.

A seventy-fourth aspect relates to the device of aspects 72-73, wherein the aerosol generator is configured to flow the fluid into the chamber.

A seventy-fifth aspect relates to the device of aspects 71-74, wherein the aerosol generator is configured to flow the fluid in a direction parallel to a trajectory of the liquid droplet.

A seventy-sixth aspect relates to the device of aspects 71-74, wherein the aerosol generator is configured to flow the fluid in a direction at an angle to a trajectory of the liquid droplet.

A seventy-seventh aspect relates to the device of aspects 72-76, wherein the chamber is generally in the shape of a cone.

A seventy-eighth aspect relates to the device of aspects 72-76, wherein the chamber is generally cylindrical.

A seventy-ninth aspect relates to the device of aspects 72-76, wherein the chamber increases in width from the opening to the second opening.

An eightieth aspect relates to the device of aspects 71-79, wherein the target is a collection bath.

An eighty-first aspect relates to the device of aspects 71-80, wherein the target is spaced apart from the fluid.

An eighty-second aspect relates to the device of aspects 71-81, wherein the droplet generator is spaced apart from the fluid.

An eighty-third aspect relates to the device of aspects 71-82, wherein the droplet generator is configured to release a plurality of droplets into the fluid, the plurality of droplets released simultaneously from the droplet generator.

An eighty-fourth aspect relates to the device of aspect 83, comprising a plurality of droplet generators, and wherein the plurality of droplets is configured to be released simultaneously from the plurality of droplet generators.

An eighty-fifth aspect relates to the device of aspects 71-84, wherein the aerosol generator is configured to introduce the fluid at a volumetric flow rate such that the fluid flows through the device at a speed identical to which the liquid droplet passes through the device.

An eighty-sixth aspect relates to the device of aspects 72-85, wherein the chamber is configured to block ultraviolet radiation.

An eighty-seventh aspect relates to the device of aspects 71-86, wherein the droplet generator comprises a mixing nozzle.

An eighty-eighth aspect relates to the device of aspects 71-87, wherein the droplet generator is an acoustophoretic printer.

An eighty-ninth aspect relates to the device of aspects 72-88, wherein the chamber is an output of a three-dimensional printer.

In addition to the features mentioned in each of the independent aspects enumerated above, some examples may show, alone or in combination, the optional features mentioned in the dependent aspects and/or as disclosed in the description above and shown in the figures.

1. A method of preparing a particle, the method comprising:
releasing a liquid droplet from a droplet generator into a first fluid comprising an aerosol comprising a crosslinking agent, the droplet comprising a formulation comprising a polymer;
contacting the liquid droplet with the crosslinking agent in the first fluid;
crosslinking the liquid droplet to form the particle comprising a crosslinked matrix; and
collecting the particle.

2. The method of claim 1, wherein the particle is a gelled particle comprising a crosslinked hydrogel matrix, and the polymer is a hydrogel precursor.

3. The method of claim 1, wherein the formulation comprises a cargo dispersed in the crosslinked matrix, the cargo being a biologic.

4. (canceled)

5. The method of claim 1, further comprising:
generating an acoustic field in the first fluid with an oscillating emitter; and
detaching the droplet from the droplet generator by acoustic forces from the acoustic field.

6. (canceled)

7. The method of claim 1, wherein the aerosol comprises an aqueous solution of the crosslinking agent.

8-9. (canceled)

10. The method of claim 1, wherein a velocity of the aerosol is the same as a velocity of the liquid droplet.

11-12. (canceled)

13. The method of claim 1, wherein the contacting, crosslinking, and/or the collecting are in the absence of ultraviolet radiation.

14. The method of claim 1, wherein the collecting is in a collection bath comprising a solution of the crosslinking agent.

15-16. (canceled)

17. The method of claim 1, wherein the particle has an average diameter of less than 250 μm.

18-22. (canceled)

23. The method of claim 3, wherein a concentration of the cargo in the particle is from about 50 mg/mL to about 500 mg/mL.

24. (canceled)

25. The method of claim 1, wherein the crosslinking agent is calcium chloride.

26-39. (canceled)

40. The method of claim 1, wherein a first portion of the polymer comprises a first moiety and a second portion of the polymer comprises a second moiety;
wherein the first moiety undergoes a click chemistry reaction with the second moiety; and
wherein the first portion and the second portion are mixed in the droplet generator.

41. The method of claim 40, wherein the first moiety is a norbornenyl group and the second moiety is a tetrazinyl group.

42. (canceled)

43. A composition, comprising:
a particle formed from a liquid droplet comprising a formulation comprising a polymer, the particle comprising a crosslinked matrix;
wherein the particle comprises a particle surface comprising a plurality of surface pores, the plurality of surface pores having an average valley depth and/or an average diameter of from about 0.1 μm to about 20 μm.

44. The composition of claim 43, wherein the particle is a gelled particle comprising a crosslinked hydrogel matrix, and the polymer is a hydrogel precursor.

45. The composition of claim 43, wherein the formulation comprises a cargo dispersed in the crosslinked matrix, the cargo being a biologic.

46-50. (canceled)

51. The composition of claim 45, wherein a concentration of the cargo in the particle is from about 50 mg/mL to about 500 mg/mL.

52. (canceled)

53. The composition of claim 43, wherein the composition is acoustophoretically printed.

54-57. (canceled)

58. The composition of claim 45, wherein the cargo is homogeneously dispersed in the crosslinked matrix.

59-70. (canceled)

71. An in-flight crosslinking device, comprising:
an aerosol generator configured to introduce into the device a fluid comprising an aerosol comprising a crosslinking agent;
a droplet generator configured to release into the fluid a liquid droplet comprising a polymer, the liquid droplet contacting the crosslinking agent; and
a target configured to receive an at least partially crosslinked particle formed from the liquid droplet.

72-89. (canceled)
